(19) United States
(12) Patent Application Publication
(10) Pub. No.: US 2003/0191158 A1
(43) Pub. Date: Oct. 9, 2003

(54) Nicotinamide derivatives and a tiotropium salt in combination for the treatment of diseases

(76) Inventor: Thomas V. Magee, Mystic, CT (US)

Correspondence Address: PFIZER INC., PATENT DEPARTMENT, MS8260-1611, EASTERN POINT ROAD, GROTON, CT, 06340, US

(21) Appl. No.: 10/360,122

(22) Filed: Feb. 6, 2003

Related U.S. Application Data
(60) Provisional application No. 60/362,154, filed on Mar. 5, 2002.

(30) Foreign Application Priority Data
Feb. 11, 2002 (GB) 0203193.8

(51) Int. Cl. C 07D 4 5/02; C07D 213/62; C07D 213/78; A61K 31/4433

(52) U.S. Cl. 514/337; 514/338; 514/350; 514/352; 546/283.7; 546/284.1; 546/298; 546/304

(57) ABSTRACT

The invention relates to a combination of a nicotinamide derivative and tiotropium or a derivative thereof, compositions containing it and the uses of, such a combination. The combination according to the present invention is useful in numerous diseases, disorders and conditions, in particular inflammatory, allergic and respiratory diseases, disorders and conditions.

This invention relates to a combination of nicotinamide derivatives of general formula:

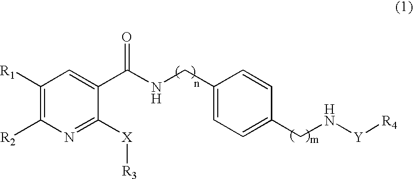

in which R₁, R₂, R₃, R₄, X, Y, n and m have the meanings indicated below, and a tiotropium salt, namely tiotropium bromide and to the uses of such combinations.

The 3′,5′-cyclic nucleotide phosphodiesterases (PDEs) comprise a large class of enzymes divided into at least eleven different families which are structurally, biochemically and pharmacologically distinct from one another. The enzymes within each family are commonly referred to as isoenzymes, or isozymes. A total of more than fifteen gene products is included within this class, and further diversity results from differential splicing and post-translational processing of those gene products. The present invention is primarily concerned with the four gene products of the fourth family of PDEs, i.e., PDE4A, PDE4B, PDE4C, and PDE4D. These enzymes are collectively referred to as being isoforms or subtypes of the PDE4 isozyme family.

The PDE4s are characterized by selective, high affinity hydrolytic degradation of the second messenger cyclic nucleotide, adenosine 3′,5′-cyclic monophosphate (cAMP), and by sensitivity to inhibition by rolipram. A number of selective inhibitors of the PDE4s have been discovered in recent years, and beneficial pharmacological effects resulting from that inhibition have been shown in a variety of disease models (see, e.g., Torphy et al., Environ. Health Perspect. ,1994, 102 Suppl. 10, p. 79-84; Duplantier et al., J. Med. Chem., 1996, 39, p. 120-125 ; Schneider et al., Pharmacol. Biochem. Behav., 1995, 50, p. 211-217; Banner and Page, Br. J. Pharmacol., 1995, 114, p. 93-98 ; Barnette et al., J. Pharmacol. Exp. Ther., 1995, 273, p. 674-679; Wright et al., Can. J. Physiol. Pharmacol., 1997, 75, p. 1001 -1008; Manabe et al., Eur. J. Pharmacol., 1997, 332, p. 97-107 and Ukita et al., J. Med. Chem., 1999, 42, p. 1088-1099). Accordingly, there continues to be considerable interest in the art with regard to the discovery of further selective inhibitors of PDE4s.

Successful results have already been obtained in the art with the discovery and development of selective PDE4 inhibitors. In vivo, PDE4 inhibitors reduce the influx of eosinophils to the lungs of allergen-challenged animals while also reducing the bronchoconstriction and elevated bronchial responsiveness occurring after allergen challenge. PDE4 inhibitors also suppress the activity of immune cells (including CD4⁺ T-lymphocytes, monocytes, mast cells, and basophils), reduce pulmonary edema, inhibit excitatory nonadrenergic noncholinergic neurotransmission (eNANC), potentiate inhibitory nonadrenergic noncholinergic neurotransmission (iNANC), reduce airway smooth muscle mitogenesis, and induce bronchodilation. PDE4 inhibitors also suppress the activity of a number of inflammatory cells associated with the pathophysiology of COPD, including monocytes/macrophages, CD4⁺ T-lymphocytes, eosinophils and neutrophils. PDE4 inhibitors also reduce vascular smooth muscle mitogenesis and potentially interfere with the ability of airway epithelial cells to generate pro-inflammatory mediators. Through the release of neutral proteases and acid hydrolases from their granules, and the generation of reactive oxygen species, neutrophils contribute to the tissue destruction associated with chronic inflammation, and are further implicated in the pathology of conditions such as emphysema. Therefore, PDE4 inhibitors are particularly useful for the treatment of a great number of inflammatory, respiratory and allergic diseases, disorders or conditions, and for wounds and some of them are in clinical development mainly for treatment of asthma, COPD, bronchitis and emphysema.

The effects of PDE4 inhibitors on various inflammatory cell responses can be used as a basis for profiling and selecting inhibitors for further study. These effects include elevation of cAMP and inhibition of superoxide production, degranulation, chemotaxis, and tumor necrosis factor alpha (TNF□) release in eosinophils, neutrophils and monocytes.

Some nicotinamide derivatives having a PDE4 inhibitory activity have already been synthetized. For example, the patent application N° WO 98/45268 discloses nicotinamide derivatives having activity as selective inhibitors of PDE4D isozyme. These selective PDE4D inhibitors are represented by the following formula:

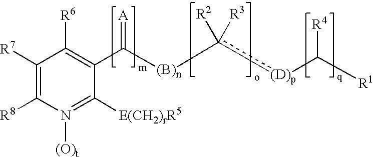

wherein in particular m and n may be equal to land p may be equal to 0, A may be oxygen, B may be NH, r may be equal to 0, E may be O, NH or S, R⁵ may be a saturated or unsaturated cyclic or bicyclic (C₃-C₇)heterocyclic group containing 1 to 4 heteroatoms and R¹ may be an aryl optionally substituted by various substituents.

The patent application N° WO 01/57036 also discloses nicotinamide derivatives which are PDE4 inhibitors useful in the treatment of various inflammatory allergic and respiratory diseases and conditions, of formula:

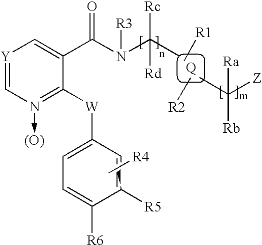

wherein in particular n is 1 or 2, m is 0 to 2, Y is ═C(R^{E})— or —[N→(O)]—, W is —O—, —S(═O)ₜ— or —N(R₃)—, Q represents various rings among which phenyl, Z is —OR₁₂, —C(═O)R₁₂ or CN and R₁₂ is choosen from alkyl, alkenyl, cycloalkyl, phenyl, benzyl and monocyclic heterocyclic moieties.

Muscarinic receptor antagonists prevent the effects resulting from passage of impulses through the parasympathetic nerves. This action results from their ability to inhibit the action of the neurotransmitter acetylcholine by blocking its binding to muscarinic cholinergic receptors. There are at least three types of muscarinic receptor subtypes. M₁ receptors are found primarily in brain and other tissue of the central nervous system, M₂ receptors are found in heart and other cardiovascular tissue, and M₃ receptors are found in smooth muscle and glandular tissues. The muscarinic receptors are located at neuroeffector sites on, e.g., smooth muscle, and in particular M₃-muscarinic receptors are located in airway smooth muscle. Consequently, anti-cholinergic agents may also be referred to as muscarinic receptor antagonists.

The parasympathetic nervous system plays a major role in regulating bronchomotor tone, and bronchoconstriction is largely the result of reflex increases in parasympathetic activity caused in turn by a diverse set of stimuli. Anti-cholinergic agents have a long history of use in the treatment of chronic airway diseases characterised by partially reversible airway narrowing such as COPD and asthma and were used as bronchodilators before the advent of epinephrine. They were thereafter supplanted by □2-adrenergic agents and methylxanthines. However, the more recent introduction of ipratropium bromide has led to a revival in the use of anti-cholinergic therapy in the treatment of respiratory diseases. However, there are muscarinic receptors on peripheral organ systems such as salivary glands and gut and therefore systemically active muscarinic receptor antagonists are limited by dry mouth and constipation. Thus the bronchodilatory and other beneficial actions of muscarinic receptor antagonists is ideally produced by an inhaled agent which has a high therapeutic index for activity in the lung compared with the peripheral compartment.

Anti-cholinergic agents also partially antagonize bronchoconstriction induced by histamine, bradykinin, or prostaglandin F_{2□}, which is deemed to reflect the participation of parasympathetic efferents in the bronchial reflexes elicited by these agents.

The anti-cholinergic tiotropium is a quaternary ammonium compound in structure, and central effects from this agent are generally lacking because such agents do not readily cross the blood-brain barrier. When agents with these characteristics are inhaled, their actions are confined almost entirely to the mouth and airways. Even when inhaled at several times the recommended dose, these agents produced little or no change in heart rate, blood pressure, bladder function, intraocular pressure, or pupillary diameter. This selectivity results from the very inefficient absorption of these agents from the lung or gastrointestinal tract. The preclinical and clinical profile of tiotropium is entirely in accord with these characteristics, with the profound difference that tiotropium has a prolonged duration of action resulting from its slow dissociation from the muscarinic M₃ receptor.

Tiotropium and derivatives thereof disclosed in EP 0 418 716 B1 constitutes quaternary nitrogen compounds having the structure of Formula (I):

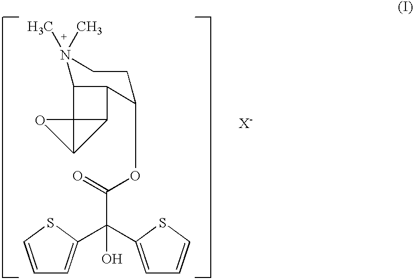

wherein X⁻ is a physiologically acceptable anion, especially bromide, and pharmaceutically acceptable solvates thereof.

Examples of suitable anions X⁻ are fluoride F⁻, chloride Cl⁻, bromide Br⁻, iodide I⁻, methanesulfonate CH₃S(═O)₂O —, ethanesulfonate CH₃CH₂S(═O)₂O-, methylsulfate CH₃O═O)₂O⁻, benzene sulfonate C₆H₅S(═O)₂O⁻, and p-toluenesulfonate 4—CH₃—C₆H₅S(═O)₂O⁻.

However, there is still a huge need for additional PDE4 inhibitors showing improved therapeutic index with possibly less adverse effects (such as for example emesis) that would exhibit an improved potency and a better toleration in combination with tiotropium or a derivative thereof.

In this context, the present invention relates to novel PDE4 inhibitors of the nicotinamide family in combination with tiotropium or a derivative thereof, namely tiotropium bromide.

The novel PDE4 inhibitors of the present invention are nicotinamide derivatives of general formula (1):

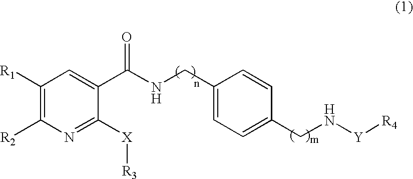

m is 0, 1, 2 or 3,

n is 0, 1, 2 or 3,

R₁ and R₂ are each a member independently selected from the group consisting of hydrogen atom, halo, cyano, (C₁-C₄)alkyl and (C₁-C₄)alkoxy,

X is —O—, —S— or —NH—,

R₃ is a member selected from the groups consisting of:

(a) phenyl, naphthyl, heteroaryl and (C₃-C₈)cycloalkyl, each optionally substituted with 1 to 3 substituents each selected from the group consisting of halo, cyano, (C₁-C₄)alkyl, (C₁-C₄)alkoxy, (C₁-C₄)thioalkyl, —C(═O)NH₂, —C(═O)NH((C₁-C₄)alkyl), hydroxy, —O—C(═O)(C₁-C₄)alkyl, —C(═O)—O—(C₁-C₄)alkyl and hydroxy(C₁-C₄)alkyl, or

(b) the bicyclic groups conforming to one of the following structures (1.1) to (1.4):

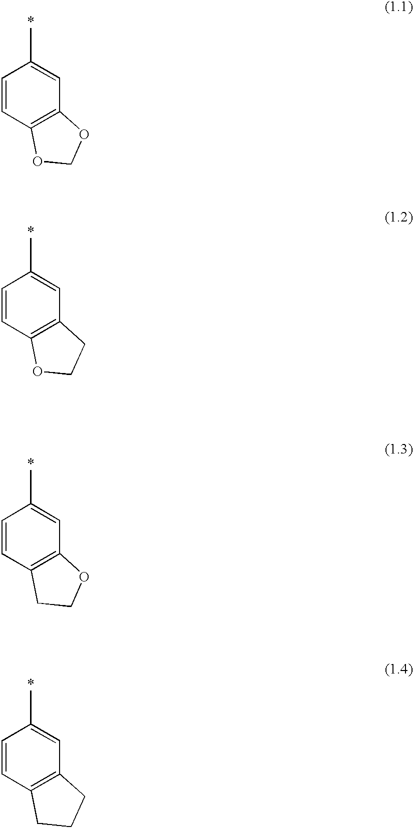

where the symbol “*” indicates the point of attachment of each partial formula (1.1) through (1.4) to the remaining portion of formula (1),

Y is a member selected from the group consisting of partial formulas (1.5) through (1.11):

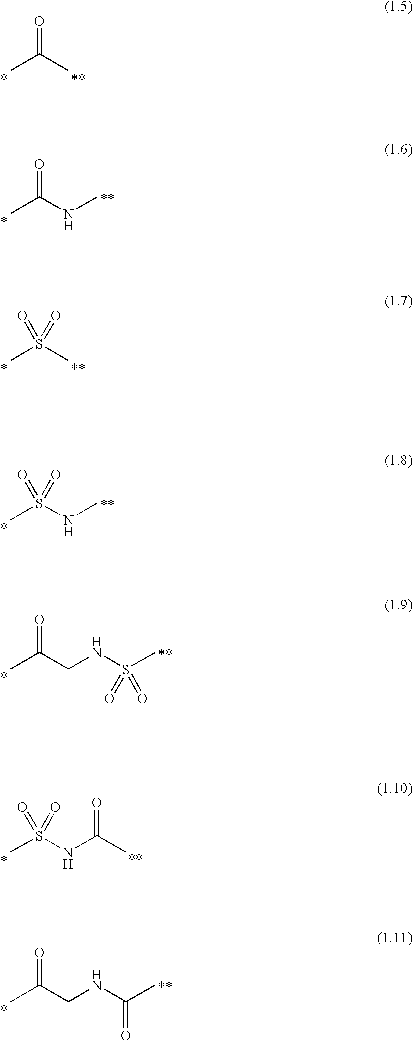

where the symbol “*” indicates the point of attachment of each partial formula (1.5) through (1.11) to the remaining portions —NH— of formula (1) and “**” indicates the point of attachment of each partial formula (1.5) through (1.11 ) to the remaining portions —R₄ of formula (1),

and R₄ is a member selected from the groups consisting of:

(a) phenyl, naphthyl and heteroaryl, each optionally substituted with 1 to 3 substituents each selected from the group consisting of carboxylic acid, C(═O)—O—(C₁-C₄)alkyl, halo, cyano, —C(═O)NH₂, (C₁-C₄)alkyl, (C₁-C₄)alkoxy, (C₁-C₄)haloalkyl, hydroxy, and hydroxy(C₁-C₄)alkyl, or

(b) (C₁-C₄)alkyl optionally substituted with a hydroxy, carboxylic acid, C(═O)—O—(C₁-C₄)alkyl, phenyl, naphthyl or heteroaryl group wherein said phenyl, naphthyl and heteroaryl are each optionally substituted with 1 to 3 substituents each selected from the group consisting of carboxylic acid, C(═O)O(C₁-C₄)alkyl, halo, cyano, —C(═O)NH₂, (C₁-C₄)alkyl or (C₁-C₄)alkoxy, (C₁-C₄)haloalkyl, hydroxy, and hydroxy(C₁-C₄)alkyl,

or, if appropriate, their pharmaceutically acceptable salts and/or isomers, tautomers, solvates, polymorphs, isotopic variations or metabolites thereof.

It has been found that these nicotinamide derivatives are inhibitors of PDE4 isoenzymes, particularly useful for the treatment of inflammatory, respiratory and allergic diseases and conditions and for the treatment of wounds by showing excellent therapeutic utility and therapeutic index.

In the here above general formula (1), halo denotes a halogen atom selected from the group consisting of fluoro, chloro, bromo and iodo in particular fluoro or chloro.

(C₁-C₄)alkyl radicals denote a straight-chain or branched group containing 1, 2, 3 or 4 carbon atoms. This also applies if they carry substituents or occur as substituents of other radicals, for example in (C₁-C₄)alkoxy radicals, (C₁-C₄)thioalkyl radicals, (C₁-C₄)haloalkyl radicals, hydroxy(C₁-C₄)alkyl radicals, C(═O)O(C₁-C₄)alkyl radicals etc. . . . Examples of suitable (C₁-C₄)alkyl radicals are methyl, ethyl, n-propyl, iso-propyl, n-butyl, iso-butyl, sec-butyl and tert-butyl. Examples of suitable (C₁-C₄)alkoxy radicals are methoxy, ethoxy, n-propyloxy, iso-propyloxy, n-butyloxy, iso-butyloxy, sec-butyloxy and tert-butyloxy. Examples of suitable (C₁-C₄)thioalkyl radicals are thiomethyl, thioethyl, thio-n-propyl, thio-iso-propyl, thio-n-butyl, thio-iso-butyl, thio-sec-butyl and thio-tert-butyl. (C₁-C₄)haloalkyl radicals are alkyl radicals substituted by halo. They can contain 1, 2, 3, 4, 5, 6 or 7 halogen atoms, if not stated otherwise. Said halo is preferably a fluoro, a chloro, a bromo or a iodo, in particular fluoro or chloro. For example in a fluoro-substituted alkyl radical, a methyl group can be present as a trifluoromethyl group. The same applies to hydroxy(C₁-C₄)alkyl radicals except that they are alkyl radicals substituted by a hydroxy group (—OH). According to a preferred embodiment of said invention, such radicals contain one hydroxy substituent. Examples of suitable hydroxy(C₁-C₄)alkyl radicals are hydroxymethyl, 1-hydroxyethyl or 2-hydroxyethyl.

(C₃-C₈)cycloalkyl radicals represent 3-membered to 8-membered saturated monocyclic rings. Examples of suitable (C₃-C₈)cycloalkyl radicals are in particular cyclopropyl, cyclobutyl, cyclopentyl, cyclohexyl, cycloheptyl and cyclooctyl. These radicasl can be optionally substituted as indicated in the definition of R₃. Examples of substituted (C₃-C₈)cycloalkyl radicals are 2-methylcyclohexyl, 3-methylcyclohexyl, 4-methylcyclohexyl, 5-methylcyclohexyl, 6-methylcyclohexyl, 2-hydroxycyclohexyl, 3-hydroxycyclohexyl, 4-hydroxycyclohexyl, 5-hydroxycyclohexyl, 6-hydroxycyclohexyl, 2-fluorocyclohexyl, 3-fluorocyclohexyl, 4-fluorocyclohexyl, 5-fluorocyclohexyl, 6-fluorocyclohexyl 2-methyl-3-hydroxycyclohexyl, 2-methyl-4-hydroxycyclohexyl, 2-hydroxy-4-methylcyclohexyl, etc. . . .

In the hereabove general formula (1), heteroaryl is a radical of a monocyclic or polycyclic aromatic system having 5 to 14 ring members, which contains 1, 2, 3, 4 or 5 heteroatom(s) depending in number and quality of the total number of ring members. Examples of heteroatoms are nitrogen (N), oxygen (O) and sulphur (S). If several heteroatoms are contained, these can be identical or different. Heteroaryl radicals can also be unsubstituted, monosubstituted or polysubstituted, as indicated in the definition of R₃ and R₄ hereabove for general formula (1) according to the present invention. Preferably the heteroaryl is a monocyclic or bicyclic aromatic radical which contains 1, 2, 3 or 4, in particular 1, 2 or 3, identical or different heteroatoms selected from the group consisting of N, O and S. Particularly preferably, the heteroaryl is a monocyclic or bicyclic aromatic radical having 5 to 10 ring members, in particular a 5-membered to 6-membered monocyclic aromatic radical which contains (i) from 1 to 4 nitrogen heteroatom(s) or (ii) 1 or 2 nitrogen heteroatom(s) and 1 oxygen heteroatom or 1 sulphur heteroatom or (iii) 1 or 2 oxygen or sulphur heteroatom(s). Examples of suitable heteroaryl radicals are the radicals derivated from pyrrole, furan, furazan, thiophene, imidazole, pyrazole, oxazole, isoxazole, thiazole, isothiazole, tetrazole, triazine, pyridine, pyrazine, pyrimidine, pyridazine, indolizine, indole, isoindole, indazole, purine, naphtyridine, phthalazine, quinoline, isoquinoline, quinoxaline, quinazoline, cinnoline, and benzo-fused derivatives of these heteroaryls, such as for example benzofuran, benzothiophene, benzoxazole, and benzothiazole. Particularly preferred are the heteroaryl radicals selected from pyrrolyl, pyrazolyl, 1,2,3-triazolyl, 1,2,4-triazolyl, tetrazolyl, oxazolyl, isoxazolyl, thiazolyl, isothiazolyl, 1,2,4-oxadiazolyl, 1,3,4-oxadiazolyl, furanyl, thienyl, pyridinyl, pyridazinyl, pyrimidinyl, and pyrazinyl. Nitrogen heteroaryl radicals can also be present as N-oxides or as quaternary salts.

In the general formula (1) according to the present invention, when a radical is mono- or poly-substituted, said substituent(s) can be located at any desired position(s). Also, when a radical is polysubstituted, said substituents can be identical or different.

The nicotinamide derivatives of the formula (1) can be prepared using conventional procedures such as by the following illustrative methods in which R₁, R₂, R₃, R₄, X, Y, n and m are as previously defined for the nicotinamide derivatives of the formula (1) unless otherwise stated.

The nicotinamide derivatives of the formula (1) may be prepared starting from a compound of formula (2):

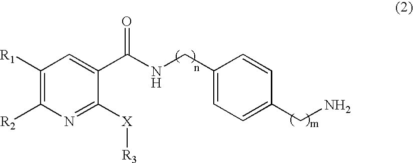

wherein R₁, R₂, X, R₃, n and m are as previously described for the nicotinamide derivatives of formula (1).

Where Y represents a group of partial formula (1.7), (1.8) or (1.10), the compounds of formula (2) may be reacted with the corresponding R₄-sulfonyl chloride derivative (R₄SO₂Cl or R₄NHSO₂Cl or R₄C(═O)NHSO₂Cl) in a suitable solvent (e.g. dichloromethane) and in the presence of an organic base (e.g. triethylamine) at a temperature ranging from 0° C. to room temperature (about 20° C.).

Where Y represents a group of partial formula (1.5), (1.9) or (1.11), the compounds of formula (2) may be reacted with the corresponding R₄-carboxylic acid derivative (R₄COOH or R₄SO₂NH—CH₂—COOH or R₄C(O)NH—CH₂—COOH) using an activating agent in the presence of a suitable solvent (e.g. dimethylformamide) and organic base (e.g. N-methylmorpholine) at room temperature. Activation of the acid may be achieved by using for example:

a) 1-hydroxybenzotriazole and 1-(3-dimethylaminopropyl)-3-ethylcarbodiimide hydrochloride,

b) carbonyldiimidazole, or

c) oxalyl chloride and dimethylformamide (with dichloromethane as the solvent).

Where Y represents a group of partial formula (1.6), the compounds of formula (2) may be reacted with carbonyldiimidazole in a suitable solvent (such as dichloromethane) and the obtained intermediate is reacted with an amine bearing the substituent R₄.

It must be emphasized that where R₃ and R₄ in the nicotinamide derivatives of formula (1) represent alkoxy substituted phenyl rings, these structures can be converted to the hydroxy analogue using certain deprotection conditions well-known to the one skilled in the art.

The compounds of general formula (2) may be prepared by removal of the protecting group “Prot” from the compounds of general formula (3):

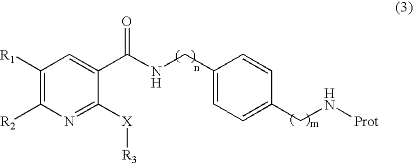

wherein R₁, R₂, X, R₃, n and m are as previously described for the nicotinamide derivatives of formula (1) and Prot is a suitable protecting group, which includes but is not limited to a benzyl group, a carbamate (e.g. tert-butyloxycarbonyl), an amide (e.g. trifluoroacetamide) or an imide (e.g. phtalimide), using deprotection conditions well-known to the one skilled in the art.

The compounds of formula (3) may be prepared as shown in scheme 1:

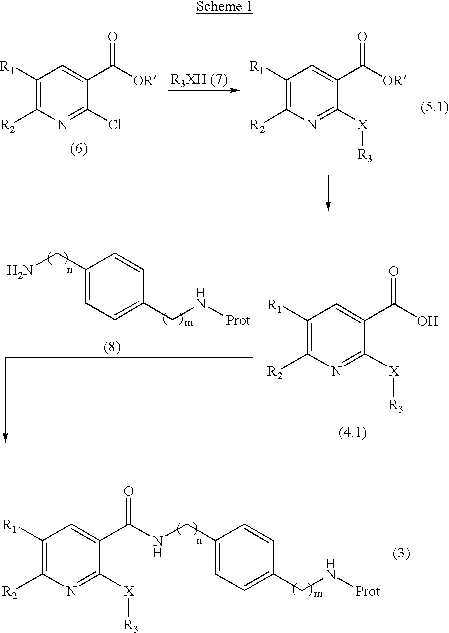

wherein R₁, R₂, X, R₃, n, m and Prot are as previously described and R′ represents a (C₁-C₄)alkyl radical.

In a typical procedure the nicotinate ester of the formula (6) may be reacted with the appropriate alcohol, thiol or amine of formula R₃XH (7) in the appropriate solvent (for example dimethylformamide or dioxan) containing a base, such as cesium carbonate, at temperatures ranging from room temperature to 100° C. to give a compound of the formula (5.1). This can be saponified with an alkali-hydroxide to give an acid of the formula (4.1) which is then converted to a compound of the formula (3) by reaction with a monoprotected diamine of the formula (8):

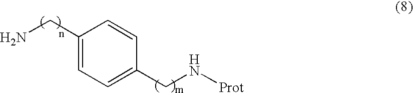

using an activating agent such as those described in one of the activation methods outlined before (i.e. a) 1-hydroxybenzotriazole and 1-(3-dimethylaminopropyl)-3-ethylcarbodiimide hydrochloride or b) carbonyldiimidazole or c) oxalyl chloride and dimethylformamide, with dichloromethane as the solvent).

According to another alternative, the compounds of formula (3) may be prepared as shown in scheme 2:

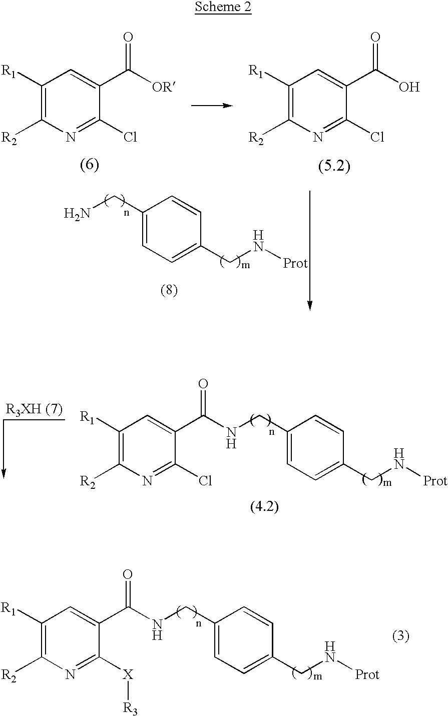

wherein R₁, R₂, X, R₃, n, m, R′ and Prot are as previously described.

In a typical procedure the nicotinate ester of the formula (6) may be hydrolysed using an alkaline metal hydroxide to a nicotinic acid of the formula (5.2), which is reacted with a monoprotected diamine of the formula (8) using one of the activation methods outlined before. The chloropyridine of the formula (4.2) obtained at the preceding step may be reacted with the appropriate alcohol, thiol or amine of formula R₃XH (7) in the appropriate solvent (for example dimethylformamide or dioxan) containing a base, such as cesium carbonate, at temperatures ranging from room temperature to 100° C.

The compounds of formula (6) and (7) are either commercial or they can be prepared by conventional procedures well known to the one skilled in the art.

The monoprotected diamine of the formula (8) may be prepared by reaction of a large excess of a diamine of formula (9):

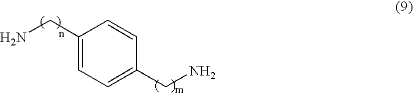

wherein m and n are as defined above, with a suitable derivatizing agent such as di-tert-butyldicarboxylate (to give the tert-butyloxycarbonyl derivative) at room temperature in a suitable solvent (such as dichloromethane).

The compounds of formula (9) are commercial or they can be easily prepared by conventional procedures well known to the one skilled in the art.

All of the above reactions and the preparations of novel starting materials using in the preceding methods are conventional and appropriate reagents and reaction conditions for their performance or preparation as well as procedures for isolating the desired products will be well-known to those skilled in the art with reference to literature precedents and the examples and preparations hereto.

For some of the,steps of the here above described process of preparation of the nicotinamide derivatives of formula (1), it can be necessary to protect the potential reactive functions that are not wished to react. In such a case, any compatible protecting radical can be used. In particular methods such as those described by T. W. GREENE (Protective Groups in Organic Synthesis, A. Wiley-Interscience Publication, 1981) or by McOMIE (Protective Groups in Organic Chemistry, Plenum Press, 1973), can be used.

Also, the nicotinamide derivatives of formula (1) as well as intermediate for the preparation thereof can be purified according to various well-known methods, such as for example crystallization or chromatography.

According to a general aspect of the present invention, the nicotinamide derivatives of the formula (1) as previously described except the compounds for which

1) m is different from 0 simultaneously with Y representing the partial formula (1.5) and R₄ representing a non-substituted (C₁-C₄)alkyl,

2) m is equal to 0 simultaneously with Y representing the partial formula (1.5) and R₄ representing a phenyl, a naphtyl or a heteroaryl each optionally substituted with 1 to 3 substituents independently selected from the group consisting of carboxylic acid, halo, cyano, (C₁-C₄)alkyl, (C₁-C₄)alkoxy, (C₁-C₄)haloalkyl, hydroxy and hydroxy(C₁-C₄)alkyl or R₄ representing a (C₁-C₄)alkyl optionally substituted with a hydroxy, carboxylic acid, or a heteroaryl, which is optionally substituted with 1 to 3 substituents independently selected from the group consisting of carboxylic acid, halo, cyano, (C₁-C₄)alkyl, (C₁-C₄)alkoxy, hydroxy and hydroxy(C₁-C₄)alkyl, and

3) m is equal to 0 simultaneously with Y representing the partial formula (1.6) and R₄ representing a phenyl or a naphtyl, each optionally substituted with 1 to 3 substituents independently selected from the group consisting of carboxylic acid, halo, cyano, (C₁-C₄)alkyl, (C₁-C₄)alkoxy, (C₁-C₄)haloalkyl, hydroxy and hydroxy(C₁-C₄)alkyl,

are preferred.

Particularly preferred are nicotinamide derivatives of the formula (1) in which

m and n are equal to 1,

R₁ and R₂ are each a member independently selected from the group consisting of hydrogen atom, halo, cyano, (C₁-C₄)alkyl and (C₁-C₄)alkoxy,

X is —O—,

R₃ is a member selected from the groups consisting of:

(a) phenyl, naphthyl, heteroaryl and (C₃-C₈)cycloalkyl, each optionally substituted with 1 to 3 substituents each selected from the group consisting of halo, cyano, (C₁-C₄)alkyl, (C₁-C₄)alkoxy, (C₁-C₄)thioalkyl, —C(═O)NH₂, —C(═O)N H((C₁-C₄)alkyl), hydroxy —O—C(═O)(C₁-C₄)alkyl, —C(═O)—O—(C₁-C₄)alkyl and hydroxy(C₁-C₄)alkyl, or

(b) the bicyclic groups conforming to one of the following structures (1.1) to (1.4):

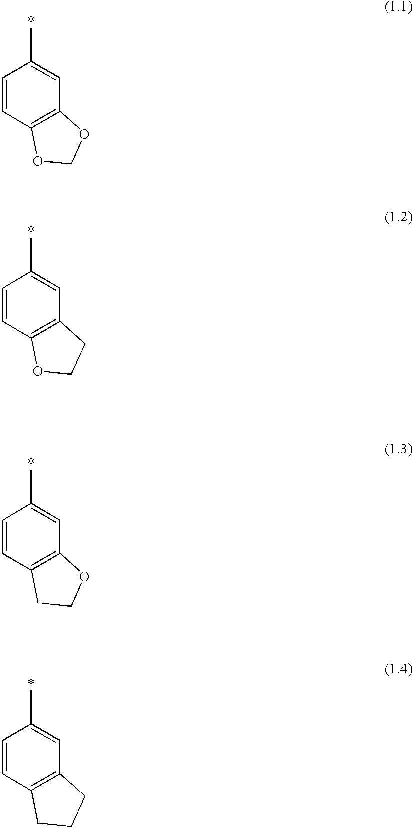

where the symbol “*” indicates the point of attachment of each partial formula (1.1) through (1.4) to the remaining portion of formula (1),

Y is a group —C(═O)— of partial formula (1.5)

and R₄ is a member selected from the groups consisting of:

(a) phenyl, naphthyl and heteroaryl, each optionally substituted with 1 to 3 substituents each selected from the group consisting of carboxylic acid, C(═O)—O—(C₁-C₄)alkyl, halo, cyano, —C(═O)NH₂, (C₁-C₄)alkyl, (C₁-C₄)alkoxy, (C₁-C₄)haloalkyl, hydroxy, and hydroxy(C₁-C₄)alkyl, or

(b) (C₁-C₄)alkyl substituted with a hydroxy, carboxylic acid, C(═O)—O—(C₁-C₄)alkyl, phenyl, naphthyl or heteroaryl group wherein said phenyl, naphthyl and heteroaryl are each optionally substituted with 1 to 3 substituents each selected from the group consisting of carboxylic acid, C(═O)O(C₁-C₄)alkyl, halo, cyano, —C(═O)NH₂, (C₁-C₄)alkyl, (C₁-C₄)alkoxy, (C₁-C₄)haloalkyl, hydroxy, and hydroxy(C₁-C₄)alkyl, or, if appropriate, their pharmaceutically acceptable salts and/or isomers, tautomers, solvates, polymorphs, isotopic variations or metabolites thereof.

More particularly preferred are the nicotinamide derivatives of the formula (1) in which:

m and n are equal to 1,

R₁ and R₂ are each a member independently selected from the group consisting of hydrogen atom, halo and methyl,

X is —O—,

R₃ is a phenyl optionally substituted with 1 to 3 substituents each selected from the group consisting of halo, cyano, (C₁-C₄)alkyl, (C₁-C₄)alkoxy, (C₁-C₄)thioalkyl, —C(═O)NH₂, —C(═O)NH((C₁-C₄)alkyl), hydroxy, —O—C(═O)(C₁-C₄)alkyl, —C(═O)—O—(C₁-C₄)alkyl and hydroxy(C₁-C₄)alkyl,

Y is a group —C(═O)— of partial formula (1.5)

and R₄ is a member selected from the groups consisting of:

(a) phenyl optionally substituted with 1 to 3 substituents each selected from the group consisting of carboxylic acid, C(═O)—O—(C₁-C₄)alkyl, halo, cyano, —C(═O)NH₂, (C₁-C₄)alkyl, (C₁-C₄)alkoxy, (C₁-C₄)haloalkyl, hydroxy, and hydroxy(C₁-C₄)alkyl, or

(b) (C₁-C₄)alkyl substituted with a hydroxy or a phenyl, wherein said phenyl is optionally substituted with 1 to 3 substituents each selected from the group consisting of carboxylic acid, C(═O)O(C₁-C₄)alkyl, halo, cyano, —C(═O)NH₂, (C₁-C₄)alkyl or (C₁-C₄)alkoxy, (C₁-C₄)haloalkyl, hydroxy, and hydroxy(C₁-C₄)alkyl,

or, if appropriate, their pharmaceutically acceptable salts and/or isomers, tautomers, solvates, polymorphs, isotopic variations or metabolites thereof.

Still more particularly preferred are the nicotinamide derivatives of the formula (1) in which:

m and n are equal to 1,

R₁ is a hydrogen atom or a fluoro and R₂ is a hydrogen atom,

X is —O—,

R₃ is a phenyl optionally substituted with a substituent selected from the group consisting of halo and —C(═O)—O—(C₁-C₄)alkyl,

Y is a group —C(═O)— of partial formula (1.5):

and R₄ is a member selected from the groups consisting of:

(a) phenyl optionally substituted with 1 to 3 substituents each selected from the group consisting of halo, (C₁-C₄)alkyl and hydroxy, or

(b) (C₁-C₄)alkyl substituted with a hydroxy or a phenyl, wherein said phenyl is optionally substituted with 1 to 3 substituents each selected from the group consisting of halo, (C₁-C₄)alkyl and hydroxy,

or, if appropriate, their pharmaceutically acceptable salts and/or isomers, tautomers, solvates, polymorphs, isotopic variations or metabolites thereof.

Particularly preferred nicotinamide derivatives of the formula (1) are as described in the Examples section hereafter, i.e.:

2-(4-Fluoro-phenoxy)-N-{4-[(2-hydroxy-3-methyl-benzoyl amino)-methyl]-benzyl}-nicotinamide,

3-(3-{4-[(3-Hydroxy-benzoylamino)-methyl]-benzyl carbamoyl}-pyridin-2-yloxy)-benzoic acid ethyl ester,

2-(4-fluoro-phenoxy)-N-{4-[(6-fluoro-2-hydroxy-benzoylamino)-methyl]-benzyl}-nicotinamide,

2-(4-fluoro-phenoxy)-N-{4-[(5-fluoro-2-hydroxy-benzoylamino)-methyl]-benzyl}-nicotinamide,

2-(4-fluoro-phenoxy)-N-{4-[(3-hydroxy-4-methyl-benzoylamino)-methyl]-benzyl}-nicotinamide,

2-(4-fluoro-phenoxy)-N-{4-[(3-hydroxy-benzoylamino)-methyl]-benzyl}-nicotinamide,

2-(4-fluoro-phenoxy)-N-{4-[(2-hydroxy-benzoylamino)-methyl]-benzyl}-nicotinamide,

2-(4-fluoro-phenoxy)-N-{4-[(4-hydroxy-benzoylamino)-methyl]-benzyl}-nicotinamide,

2-(4-fluoro-phenoxy)-N-{4-[(2-hydroxy-4-methyl-benzoylamino)-methyl]-benzyl}-nicotinamide,

2-(4-fluoro-phenoxy)-N-{4-[(3-hydroxy-2-methyl-benzoylamino)-methyl]-benzyl}-nicotinamide,

2-(4-fluoro-phenoxy)-N-{4-[(2-hydroxy-5-methyl-benzoylamino)-methyl]-benzyl}-nicotinamide,

5-fluoro-2-(4-fluoro-phenoxy)-N-{4-[(2-hydroxy-benzoylamino)-methyl]-benzyl}-nicotinamide,

5-fluoro-2-(4-fluoro-phenoxy)-N-{4-[(2-hydroxy-acetyl-amino)-methyl]-benzyl}-nicotinamide,

5-fluoro-2-(4-fluoro-phenoxy)-N-{4-[(4-hydroxy-benzoylamino)-methyl]-benzyl}-nicotinamide,

3-(3-{4-[(3-hydroxy-benzoylamino)-methyl]-benzylcarbamoyl}-pyridin-2-yloxy)-benzoic acid ethyl ester,

3-(3-{4-[(2-hydroxy-phenacetyl-amino)-methyl]-benzylcarbamoyl}-pyridin-2-yloxy)-benzoic acid ethyl ester,

3-(3-{4-[(3-hydroxy-phenacetyl-amino)-methyl]-benzylcarbamoyl}-pyridin-2-yloxy)-benzoic acid ethyl ester,

3-(3-{4-[(4-hydroxy-phenacetyl-amino)-methyl]-benzylcarbamoyl}-pyridin-2-yloxy)-benzoic acid ethyl ester.

The nicotinamide derivatives of formula (1) may also be optionally transformed in pharmaceutically acceptable salts. In particular, these pharmaceutically acceptable salts of the nicotinamide derivatives of the formula (1) include the acid addition and the base salts thereof.

Suitable acid addition salts are formed from mineral or organic non-toxic acids which form non-toxic salts. Suitable examples of these acid addition salts are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases, which form non-toxic salts, such as alkali metal salts, earth metal salts or addition salts with ammonia and physiologically tolerable organic amines. Suitable examples of these base salts are the sodium, potassium, aluminium, calcium, magnesium, zinc or ammonium salts as well as addition salts with triethylamine, ethanolamine, diethanolamine, trimethylamine, methylamine, propylamine, diisopropylamine, N,N-dimethylethanolamine, benzylamine, dicylohexylamine, N-benzyl-β-phenethylamine, N,N′-dibenzylethylenediamine, diphénylènediamine, quinine, choline, arginine, lysine, leucine, dibenzylamine, tris(2-hydroxyethyl)amine, or α,α,α-tris(hydroxymethyl)methylamine.

Compounds, which contain both acidic groups and basic groups can also be present in the form of internal salts or betaines, which are also included by the present invention. For a review on suitable salts see Berge et al., J. Pharm. Sci., 1977, 66, p. 1-19.

Salts can generally be obtained from the nicotinamide derivatives of the formula (1) according to customary procedures known to the person skilled in the art, for example by combining with an organic or inorganic acid or base solvent or dispersant, or alternatively from other salts by anion exchange or cation exchange. The salt may precipitate from solution and be collected by filtration or may be recovered by evaporation of the solvent.

The nicotinamide derivatives of the formula (1) can also be present in stereoisomeric forms. If the nicotinamide derivatives of the formula (1) contain one or more centers of asymmetry, these can independently of one another have the (S) configuration or the (R) configuration. The invention includes all possible stereoisomers of the nicotinamide derivatives of the formula (1), for example enantiomers and diastereomers, and mixtures of two or more stereoisomeric forms, for example mixtures of enantiomers and/or diastereomers, in all ratios. The invention thus relates to enantiomers in enantiomerically pure form, both as levorotatory and dextrorotatory antipodes, in the form of racemates and in the form of mixtures of the two enantiomers in all ratios. The invention likewise relates to diastereomers in diastereomerically pure form and in the form of mixtures in all ratios. In the presence of cis/trans isomerism, the invention relates to both the cis form and the trans form and mixtures of these forms in all ratios. Individual stereoisomers can be prepared, if desired, by use of stereochemically homogeneous starting substances in the synthesis, by stereoselective synthesis or by separation of a mixture according to customary methods, for example by chromatography, crystallization or by chromatography on chiral phases. If appropriate, derivatization can be carried out before separation of stereoisomers. A stereoisomer mixture can be separated at the stage of the nicotinamide derivatives of the formula (1) or at the stage of a starting substance or of an intermediate in the course of the synthesis.

The compounds of the formula (1) according to the invention can moreover contain mobile hydrogen atoms, i.e. be present in various tautomeric forms. The present invention also relates to all tautomers of the compounds of the formula (1).

The present invention furthermore includes other types of derivatives of nicotinamide derivatives of the formula (1), for example, solvates such as hydrates and polymorphs, i.e. the various different crystalline structures of the nicotinamide derivatives according to the present invention.

The present invention also includes all suitable isotopic variations of the nicotinamide derivatives of the formula (1) or a pharmaceutically acceptable salt thereof. An isotopic variation of the nicotinamide derivatives of the formula (1) or a pharmaceutically acceptable salt thereof is defined as one in which at least one atom is replaced by an atom having the same atomic number but an atomic mass different from the atomic mass usually found in nature. Examples of isotopes that can be incorporated into the nicotinamide derivatives of the formula (1) and pharmaceutically acceptable salts thereof include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the nicotinamide derivatives of the formula (1) and pharmaceutically acceptable salts thereof, for example, those in which a radioactive isotope such as ³H or ¹⁴C is incorporated, are useful in drug and/or substrate tissue distribution studies. Tritiated, i.e. ³H, and carbon-14, i.e. ¹⁴C, isotopes are particularly preferred for their ease of preparation and detectability. Further, substitution with isotopes such as deuterium, i.e. ²H, may afford certain therapeutic advantages resulting from greater metabolic stability, for example, increased in vivo half-life or reduced dosage requirements and hence may be preferred in some circumstances. Isotopic variations of the nicotinamide derivatives of the formula (1) and pharmaceutically acceptable salts thereof of this invention can generally be prepared by conventional procedures such as by the illustrative methods or by the preparations described in the Examples and Preparations sections hereafter using appropriate isotopic variations of suitable reagents.

If appropriate, the present invention also concerns the active metabolites of the nicotinamide derivatives of the formula (1), i.e. the derivatives which are formed during the cellular metabolism and that are active on organism. For example, such metabolites can be glucuronide derivatives, N-oxide derivatives or sulfonate derivatives of the compounds of the formula (1).

According to a further aspect, the present invention concerns mixtures of nicotinamide derivatives of the formula (1), as well as mixtures with or of their pharmaceutically acceptable salts, solvates, polymorphs, isomeric forms, metabolites and/or isotope forms.

According to the present invention, all the here above mentioned forms of the nicotinamide derivatives of formula (1) except the pharmaceutically acceptable salts (i.e. said solvates, polymorphs, isomeric forms, tautomers, metabolites and isotope forms), are defined as “derived forms” of the nicotinamide derivatives of formula (1) in what follows (including the claims).

The combinations of the present invention may be prepared using methodology, which is well understood by the artisan of ordinary skill. Where the combinations of the present invention are simple aqueous and/or other solvent solutions, the various components of the overall composition are brought together in any practical order, which will be dictated largely by considerations of convenience. Those components having reduced water solubility, but sufficient solubility in the same co-solvent with water, may all be dissolved in said co-solvent, after which the co-solvent solution will be added to the water portion of the carrier whereupon the solutes therein will become dissolved in the water. To aid in this dispersion/solution process, a surfactant may be employed.

The combination of the nicotinamide derivatives of formula (1), their pharmaceutically acceptable salts and/or derived forms, with tiotropium or a derivative thereof are suitable for the therapy and prophylaxis of numerous disorders in which the PDE4 enzymes and the muscarinic receptors are involved, in particular the inflammatory disorders, allergic disorders and respiratory diseases. The nicotinamide derivatives of formula (1) and their pharmaceutically acceptable salts and derived forms as mentioned above with tiotropium or a derivative thereof can be administered according to the invention to animals, preferably to mammals, and in particular to humans, as pharmaceuticals for therapy or prophylaxis. They can be administered per se, or in the form of pharmaceutical preparations, which permit administration thereof to the mammal to be treated and which contain in addition customary pharmaceutically innocuous excipients and/or additives.

Thus, the present invention also relates to compositions containing an efficacious dose of a combination of at least one nicotinamide derivative of formula (1) and/or their pharmaceutically acceptable salts and/or derived forms, and tiotropium or a derivative thereof as defined above in addition to customary pharmaceutically innocuous excipients and/or additives. Such compositions are prepared according to well-known methods compatible with the standard pharmaceutical practice. Said compositions generally contain from 0.5% to 60% in weight of the active compound and from 40 % to 99.5 % in weight of excipients and/or additives. According to the present invention, said excipients and/or additives are agents well known to the artisan for providing favourable properties in the final pharmaceutical composition. Typical excipients and/or additives include, but are by no mean limited to, acidifying and alkalizing agents, aerosol propellants, anti-microbial agents (including anti-bacterial, anti-fungal and anti-protozoal agents), antioxidants, buffering agents, chelating agents, dermatologically active agents, dispersing agents, suspending agents, emollients, emulsifying agents, penetration enhancers, preservatives, sequestering agents, solvents, stabilizers, stiffening agents, sugars, surfactants and flavouring agents. Furthermore, said compositions are prepared in a form compatible for the intended route of administration, which is used for any given patient, as well as appropriate to the disease, disorder or condition for which any given patient is being treated. Suitable routes of administration that can be envisaged include intranasal and pulmonary routes.

The combination of the nicotinamide derivatives of the formula (1), their pharmaceutically acceptable salts and/or their derived forms with tiotropium or a derivative thereof are preferably administered intra-nasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a nicotinamide derivative of the formula (1) and a suitable powder base such as lactose or starch.

Aerosol or dry powder formulations are preferably arranged so that each metered dose or “puff” contains from 1 μg to 4000 μg of a nicotinamide derivative of the formula (1) for delivery to the patient. The overall daily dose with an aerosol will be in the range of from 1 μg to 20 mg, which may be administered in a single dose or, more usually, in divided doses throughout the day.

The various pharmaceutical formulations as decribed here above are also detailed in “Pharmacie galénique” from A. Lehir (Ed. Mason, 1992, 2^{nd} edition).

The physician in any event will determine the actual dosage which will be most suitable for any individual patient and it will vary with the age, weight, health state and sex of the patient as well as the severity of the disease, disorder or condition to treat, the optional combination with other treatment(s), the response of the particular patient and in general any factor peculiar to the concerned disease, disorder or condition and to the patient. Thus, the daily dose among men may usually contain from 50 mg to 5 g of active compounds for administration singly or two or more at a time, as appropriate. There can, of course, be individual instances where higher or lower dosage ranges are merited and such are within the scope of this invention.

According to the present invention, the compositions of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. α-, β- and γ-cyclodextrins are most commonly used and suitable examples are described in WO-A-91/11172, WO-A-94/02518 and WO-A-98/55148.

As used herein, the terms “in combination with” is intended to mean, and does refer to and include the following:

simultaneous administration of such combination of nicotinamide derivative(s) and therapeutic agent(s) to a patient in need of treatment, when such components are formulated together into a single dosage form which releases said components at substantially the same time to said patient,

substantially simultaneous administration of such combination of nicotinamide derivative(s) and therapeutic agent(s) to a patient in need of treatment, when such components are formulated apart from each other into separate dosage forms which are taken at substantially the same time by said patient, whereupon said components are released at substantially the same time to said patient,

sequential administration of such combination of nicotinamide derivative(s) and therapeutic agent(s) to a patient in need of treatment, when such components are formulated apart from each other into separate dosage forms which are taken at consecutive times by said patient with a significant time interval between each administration, whereupon said components are released at substantially different times to said patient; and

sequential administration of such combination of nicotinamide derivative(s) and therapeutic agent(s) to a patient in need of treatment, when such components are formulated together into a single dosage form which releases said components in a controlled manner whereupon they are concurrently, consecutively, and/or overlappingly administered at the same and/or different times by said patient.

It is to be appreciated that all references herein to treatment include curative, palliative and prophylactic treatment.

The nicotinamide derivatives of formula (1) inhibit the PDE4 isozyme and thereby have a wide range of therapeutic applications, as described further below, because of the essential role, which the PDE4 family of isozymes plays in the physiology of all mammals. The enzymatic role performed by the PDE4 isozymes is the intracellular hydrolysis of adenosine 3′,5′-monophosphate (cAMP) within pro-inflammatory leukocytes. cAMP, in turn, is responsible for mediating the effects of numerous hormones in the body, and as a consequence, PDE4 inhibition plays a significant role in a variety of physiological processes. There is extensive literature in the art describing the effects of PDE inhibitors on various inflammatory cell responses, which in addition to cAMP increase, include inhibition of superoxide production, degranulation, chemotaxis and tumor necrosis factor (TNF) release in eosinophils, neutrophils and monocytes.

Therefore, a further aspect of the present invention relates to the use of the combinations of the instant invention in the treatment of diseases, disorders, and conditions in which the PDE4 isozymes and the muscarinic receptors are involved. More specifically, the present invention also concerns the combination of the invention, for use in the treatment of diseases, disorders, and conditions selected from the group consisting of:

asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma and wheezy infant syndrome,

chronic or acute bronchoconstriction, chronic bronchitis, small airways obstruction, and emphysema,

obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated therewith, COPD that is characterized by irreversible, progressive airways obstruction, adult respiratory distress syndrome (ARDS) and exacerbation of airways hyper-reactivity consequent to other drug therapy,

pneumoconiosis of whatever type, etiology, or pathogenesis, in particular pneumoconiosis that is a member selected from the group consisting of aluminosis or bauxite workers' disease, anthracosis or miners' asthma, asbestosis or steam-fitters' asthma, chalicosis or flint disease, ptilosis caused by inhaling the dust from ostrich feathers, siderosis caused by the inhalation of iron particles, silicosis or grinders' disease, byssinosis or cotton-dust asthma and talc pneumoconiosis,

bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,

bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis,

seasonal allergic rhinitis or perennial allergic rhinitis or sinusitis of whatever type, etiology, or pathogenesis, in particular sinusitis that is a member selected from the group consisting of purulent or nonpurulent sinusitis, acute or chronic sinusitis and ethmoid, frontal, maxillary, or sphenoid sinusitis,

an eosinophil-related disorder of whatever type, etiology, or pathogenesis, in particular an eosinophil-related disorder that is a member selected from the group consisting of eosinophilia, pulmonary infiltration eosinophilia, Loffler's syndrome, chronic eosinophilic pneumonia, tropical pulmonary eosinophilia, bronchopneumonic aspergillosis, aspergilloma, granulomas containing eosinophils, allergic granulomatous angiitis or Churg-Strauss syndrome, polyarteritis nodosa (PAN) and systemic necrotizing vasculitis,

pulmonary hypertension of whatever type, etiology or pathogenesis including primary pulmonary hypertension/essential hypertension, pulmonary hypertension secondary to congestive heart failure, pulmonary hypertension secondary to chronic obstructive pulmonary disease, pulmonary venous hypertension, pulmonary arterial hypertension and hypoxia-induced pulmonary hypertension, and

infection, especially infection by viruses wherein such viruses increase the production of TNF-α in their host, or wherein such viruses are sensitive to upregulation of TNF-α in their host so that their replication or other vital activities are adversely impacted, including a virus which is a member selected from the group consisting of HIV-1, HIV-2, and HIV-3, cytomegalovirus (CMV), influenza, adenoviruses and Herpes viruses including Herpes zoster and Herpes simplex.

A still further aspect of the present invention also relates to the use of the combinations of the invention, for the manufacture of a drug having a PDE4 inhibitory activity and an anti-muscarinic activity. In particular, the present inventions concerns the use of the combinations of the invention, for the manufacture of a drug for the treatment of inflammatory, respiratory and allergic diseases, disorders, and conditions and more precisely for the treatment of diseases, disorders, and conditions that are listed above.

As a consequence, the present invention provides a particularly interesting method of treatment of a mammal, including a human being, with a combination of a PDE4 inhibitor and tiotropium including treating said mammal with an effective amount of a combination of the invention. More precisely, the present invention provides a particularly interesting method of treatment of a mammal, including a human being, to treat an inflammatory, respiratory and allergic disease, disorder or condition, including treating said mammal with an effective amount of a combination of a nicotinamide derivative of formula (1), its pharmaceutically acceptable salts and/or derived forms with tiotropium or a derivative thereof.

The following examples illustrate the preparation of the nicotinamide derivatives of the formula (1):

### EXAMPLE 1

2-(4-Fluoro-phenoxy)-N-{4-[(2-hydroxy-3-methyl-benzoyl amino)-methyl]-benzyl}-nicotinamide

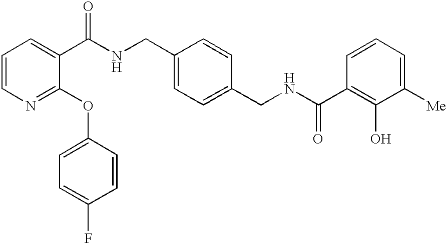

A solution of 2-Hydroxy-3-methylbenzoic acid (118 mg, 0.773 mmol), 1-hydroxybenzotriazole (157 mg, 1.16 mmol), 1-(3-dimethylaminopropyl)-3-ethylcarbodiimide hydrochloride (193 mg, 1.01 mmol), N-(4-aminomethyl-benzyl)-5-fluoro-2-(4-fluoro-phenoxy)-nicotinamide hydrochloride (300 mg, 0.773 mmol) (see Preparation 3) and N-methyl morpholine (0.17 ml, 1.55 mmol) in N,N-dimethylformamide (6 ml) was stirred under nitrogen at room temperature for 18 hours. The mixture was then partitioned between ethyl acetate (10 ml) and water (10 ml). The organic phase was separated, washed with a saturated aqueous solution of sodium chloride (10 ml) and dried over anhydrous magnesium sulphate. The solvent was then removed in vacuo and the residue was triturated with diethylether (3-fold 10 ml) giving 2-(4-fluoro-phenoxy)-N-{4-[(2-hydroxy-3-methyl-benzoylamino)-methyl]-benzyl}-nicotinamide (80 mg) as an off-white foam.

¹H NMR (300 MHz, DMSO-d⁶): □=13.11 (1H, s), 8.32-8.42 (1H, m), 8.15-8.21 (1H, m), 8.08-8.14 (1H, d), 7.66-7.75 (1H, m), 7.10-7.60 (10H, m), 6.73-6.81 (1H, t), 4.37-4.56 (4H, m), 2.16 (3H, s) ppm.

LRMS (thermospray): m/z [M+H]⁺ 486, [M+NH₄]⁺ 503

### EXAMPLES 2-15

The compounds of the following tabulated examples (Table 1) of the general formula:

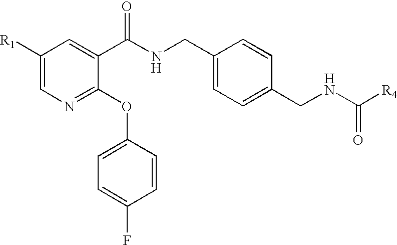

were prepared by a similar method to that of Example 1 using the appropriate amine and carboxylic acid as the starting material.

TABLE 1

| | Starting | | |
|---|---|---|---|
| | Amine Prep. | | |
| Example N° | N° | R₁ | R₄ |
| 2 | 3 | H | 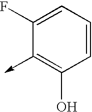 |
| 3 | 3 | H | 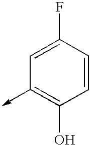 |
| 4 | 3 | H | 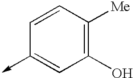 |
| 5 | 3 | H | 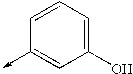 |
| 6 | 3 | H | 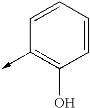 |
| 7 | 3 | H | 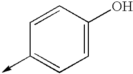 |
| 8 | 3 | H | 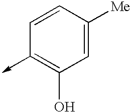 |
| 9 | 3 | H | 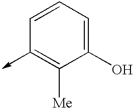 |
| 10 | 3 | H | 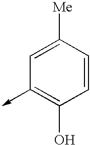 |
| 11^{1,2} | 6 | F | 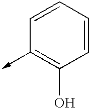 |
| 12¹ | 6 | F | 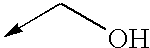 |
| 13^{1,2} | 6 | F | 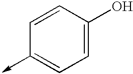 |

¹The organic phase was washed sequentially with water and a saturated aqueous solution of sodium hydrogen carbonate in the work-up procedure.
²The compound was purified by flash column chromatography on silica gel eluting with a solvent gradient of pentane:ethyl acetate (95:5 changing to 70:30, by volume).

¹ The organic phase was washed sequentially with water and a saturated aqueous solution of sodium hydrogen carbonate in the work-up procedure.

² The compound was purified by flash column chromatography on silica gel eluting with a solvent gradient of pentane: ethyl acetate (95:5 changing to 70:30, by volume).

### EXAMPLE 2

¹H NMR (300 MHz, DMSO-d⁶): □=11.28 (1H, s), 8.86-8.92 (1H, m), 8.73-8.85 (1H, m), 8.10-8.22 (2H, m), 7.16-7.36 (10H, m), 6.63-6.76 (2H, m), 4.47-4.56 (2H, d), 4.40-4.46 (2H, d) ppm.

LRMS (thermospray): m/z [M+H]⁺ 490, [M+NH₄]⁺ 507

### EXAMPLE 3

¹H NMR (300 MHz, DMSO-d⁶): □=12.20 (1H, s), 9.23-9.11 (1H, m), 8.83-8.92 (1H, m), 8.17-8.21 (1H, m), 8.10-8.15 (2H, m), 7.67-7.75 (1H, m), 7.18-7.33 (10H, m), 6.86-6.96 (2H, m), 4.42-4.51 (4H, m) ppm.

LRMS (thermospray): m/z [M+H]⁺ 490, [M+NH₄]⁺ 507

### EXAMPLE 4

¹H NMR (300 MHz, DMSO-d⁶): □=9.46 (1H, s), 8.83-8.92 (1H, t), 8.75-8.82 (1H, t), 8.16-8.20 (1H, d), 8.09-8.14 (1H, d), 7.15-7.32 (11H, m), 7.06-7.14 (1H, d), 4.43-4.51 (2H, d), 4.34-4.42 (2H, d), 2.13 (3H, s) ppm.

LRMS (thermospray): m/z [M+H]⁺ 486, [M+NH₄]⁺ 503

### EXAMPLE 5

¹H NMR (300 MHz, CDCl₃): □=8.54-8.61 (1H, d), 8.23-8.32 (1H, m), 8.17-8.23 (1H, m), 7.61-7.80 (1H, m), 7.35-7.40 (2H, m), 7.02-7.30 (9H, m), 6.90-7.00 (1H, m), 6.70-6.78 (1H, m), 4.60-4.70 (2H, d), 4.48-4.59 (2H, d) ppm.

LRMS (thermospray): m/z [M+H]⁺ 472, [M+NH₄]⁺ 489.

### EXAMPLE 6

¹H NMR (300 MHz, CDCl₃): □=12.02-12.50 (1H, brs), 8.53-8.70 (1H, brs), 8.10-8.26 (2H, brs), 6.92-7.50 (12H, m), 6.63-6.88 (2H, m), 4.56-4.77 (4H, 2xm) ppm.

LRMS (thermospray): m/z [M+H]⁺ 472, [M+NH₄]⁺ 489.

### EXAMPLE 7

¹H NMR (300 MHz, DMSO-d⁶) □=9.93 (1H, s), 8.84-8.91 (1H, t), 8.68-8.76 (1H, t), 8.17-8.21 (1H, m), 8.10-8.15 (1H, d), 7.69-7.76 (2H, d), 7.18-7.33 (9H, m), 6.74-6.81 (2H, d), 4.44-4.52 (2H, d), 4.37-4.43 (2H, d) ppm.

LRMS (thermospray): m/z [M+H]⁺ 472, [M+NH₄]⁺ 489.

### EXAMPLE 8

¹H NMR (300 MHz, DMSO-d⁶): □=12.45-12.60 (1H, brs), 9.17-9.25 (1H, t), 8.84-8.92 (1H, t), 8.16-8.20 (1H, d), 8.09-8.14 (1H, d), 7.72-7.78 (1H, d), 7.16-7.34 (9H, m), 6.67-6.73 (2H, m), 4.40-4.58 (4H, 2xd), 2.25 (3H, s) ppm.

LRMS (thermospray): m/z [M+H]⁺ 486, [M+NH₄]⁺ 503

### EXAMPLE 9

¹H NMR (300 MHz, DMSO-d⁶): □=9.43 (1H, s), 8.87-8.96 (1H, t), 8.60-8.67 (1H, t), 8.16-8.21 (1H, d), 8.10-8.15 (1H, d), 7.28-7.34 (1H, d), 7.20-7.28 (8H, m), 6.96-7.03 (1H, t), 6.80-6.86 (1H, d), 6.73-6.77 (1H, d), 4.47-4.54 (2H, d), 4.34-4.40 (2H, d), 2.07 (3H, s) ppm.

LRMS (thermospray): m/z [M+H]⁺ 486, [M+NH₄]⁺ 503

### EXAMPLE 10

¹H NMR (300 MHz, DMSO-d⁶): □=12.23 (1H, s), 9.18-9.26 (1H, t), 8.82-8.92 (1H, t), 8.15-8.20 (1H, d), 8.10-8.15 (1H, d), 7.69 (1H, s), 7.12-7.34 (10H, m), 6.77-6.81 (1H, d), 4.42-4.54 (4H, 2xd), 2.20 (3H, s) ppm.

LRMS (thermospray): m/z [M+H]⁺ 486, [M+NH₄]⁺ 503

### EXAMPLE 11

¹H NMR (400 MHz, DMSO-d⁶): □=9.24-9.31 (1H, m), 8.92-9.00 (1H, m), 8.18-8.20 (1H, d), 8.02-8.07 (1H, dd), 7.83-7.87 (1H, d), 7.35-7.40 (1H, t), 7.18-7.35 (8H, m), 6.83-6.92 (2H, t), 4.42-4.56 (4H, m) ppm.

LRMS (electrospray): m/z [M+Na]⁺ 512, [M−H]⁺ 488.

### EXAMPLE 12

¹H NMR (400 MHz, DMSO-d⁶): □=8.93-9.00 (1H, m), 8.13-8.22 (2H, m), 8.02-8.08 (1H, m), 7.14-7.27 (8H, m), 5.38-5.43 (1H, t), 4.43-4.51 (2H, d), 4.21-4.27 (2H, d), 3.79-3.84 (2H, d) ppm.

LRMS (electrospray): m/z [M−H]⁺ 426.

### EXAMPLE 13

¹H NMR (400 MHz, DMSO-d⁶): □=9.89 (1H, s), 8.90-8.98 (1H, t), 8.64-8.73 (1H, t), 8.19-8.21 (1H, d), 8.02-8.06 (1H, dd), 7.70-7.77 (2H, d), 7.24-7.30 (2H, d), 7.17-7.23 (6H, d), 6.73-6.79 (2H, d), 4.42-4.48 (2H, d), 4.36-4.40 (2H, d) ppm.

LRMS (electrospray): m/z [M+Na]⁺ 512, [M−H]⁺ 488.

### EXAMPLE 14

3-(3-{4-[(3-Hydroxy-benzoylamino)-methyl]-benzyl carbamoyl}-Pyridin-2-yloxy)-benzoic acid ethyl ester

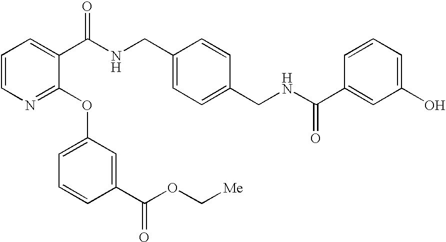

3-Hydroxy-benzoic acid (27 mg, 0.19 mmol), 1-hydroxybenzotriazole (31 mg, 0.23 mmol) and 1-(3-dimethylaminopropyl)-3-ethylcarbodiimide hydrochloride (45 mg, 0.23 mmol) were added to a solution of 3-[3-(4-aminomethyl-benzylcarbamoyl)-pyridin-2-yloxy]-benzoic acid ethyl ester hydrochloride (100 mg, 0.19 mmol) (see Preparation 9) and N-methyl morpholine (0.11 ml, 0.97 mmol) in N,N-dimethylformamide (15 ml). The reaction mixture was stirred at room temperature under a nitrogen atmosphere for 18 hours, concentrated in vacuo and the residue partitioned between dichloromethane (20 ml) and water (20 ml). The organic phase was separated, washed with a saturated aqueous solution of sodium chloride (20 ml), dried over anhydrous magnesium sulphate and concentrated under reduced pressure. The residue was then purified by flash column chromatography on silica gel eluting with a solvent gradient of dichloromethane: methanol (99:1 changing to 98:1, by volume) giving 3-(3-{4-[(3-hydroxy-benzoylamino)-methyl]-benzylcarbamoyl}-pyridin-2-yloxy)-benzoic acid ethyl ester (45 mg) as an off-white foam.

¹H NMR (400 MHz, CDCl₃): □=8.54-8.60 (1H, d), 8.21-8.38 (2H, t+brs), 8.17-8.20 (1H, d), 7.86-7.92 (1H, d) , 7.78 (1H, s), 7.41-7.48 (1H, t), 7.28-7.37 (2H, m), 7.08-7.26 (6H, m), 6.86-6.95 (2H, m), 4.61-4.67 (2H, d), 4.45-4.53 (2H, d), 4.30-4.37 (2H, quart), 1.31-1.38 (3H, t) ppm.

LRMS (electrospray): m/z [M+H]⁺ 526, [M+Na]⁺ 548, [M−H]⁺ 524.

### EXAMPLES 15-18

The compounds of the following tabulated examples (Table 2) of the general formula:

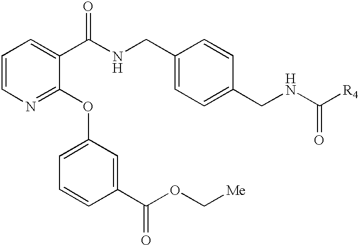

were prepared by a similar method to that of Example 14 using the appropriate carboxylic acid starting material.

TABLE 2

| | Starting Amine | |
|---|---|---|
| Example N° | Prep. N° | R₄ |
| 15 | 9 | 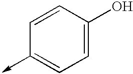 |
| 16 | 9 | 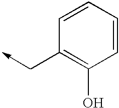 |
| 17 | 9 | 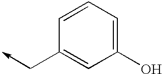 |
| 18 | 9 | 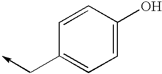 |

### EXAMPLE 15

¹H NMR (400 MHz, CDCl₃): □=8.90-9.10 (1H, brs), 8.49-8.53 (1H, d), 8.28-8.34 (1H, m), 8.13-8.16 (1H, d), 7.87-7.92 (1H, d), 7.77 (1H, s), 7.53-7.59 (2H, d), 7.40-7.47 (1H, t), 7.28-7.33 (1H, m), 7.14-7.26 (5H, m, partially masked by solvent), 7.08-7.13 (1H, t), 6.75-6.81 (1H, t), 6.66-6.73 (2H, d), 4.58-4.66 (2H, d), 4.46-4.52 (2H, d), 4.28-4.34 (2H, quartet), 1.31-1.38 (3H, t) ppm.

LRMS (electrospray): m/z [M+H⁺ 526, [M+Na]⁺ 548, [M−H]⁺ 524.

### EXAMPLE 16

¹H NMR (400 MHz, CDCl₃): □=9.61 (1H, s), 8.54-8.60 (1H, d), 8.14-8.21 (2H, m), 7.91-7.96 (1H, d), 7.72-7.74 (1H, m), 7.43-7.49 (1H, t), 7.29-7.33 (1H, d), 7.19-7.24 (2H, m), 7.08-7.18 (4H, m), 6.90-7.00 (2H, m), 6.73-6.80 (2H, m), 4.58-4.63 (2H, d), 4.29-4.39 (4H, m), 3.56 (2H, s), 1.35-1.41 (3H, t) ppm.

LRMS (electrospray): m/z [M+H]⁺ 540, [M+Na]⁺ 562, [M−H]⁺ 538.

### EXAMPLE 17

¹H NMR (400 MHz, CDCl₃): □=8.52-8.59 (1H, d), 8.19-8.25 (1H, m), 8.16-8.19 (1H, d), 7.90-7.94 (1H, d), 7.78 (1H, s), 7.44-7.49 (1H, t), 7.28-7.32 (1H, d), 7.18-7.23 (2H, d), 7.04-7.18 (4H, m), 6.64-6.73 (3H, m), 6.28-6.35 (1H, m), 4.58-4.66 (2H, d), 4.26-4.38 (4H, m), 3.42 (2H, s), 1.33-1.38 (3H, t), ppm.

LRMS (electrospray) : m/z [M−H]⁺ 538.

### EXAMPLE 18

¹H NMR (400 MHz, CDCl₃): □=8.55-8.61 (1H, d), 8.18-8.23 (1H, m), 8.15-8.18 (1H, d), 7.90-7.94 (1H, d), 7.78 (1H, s), 7.43-7.49 (1H, t), 7.26-7.30 (1H, d), 7.18-7.25 (2H, m), 7.04-7.17 (3H, m), 6.95-7.01 (2H, d), 6.64-6.75 (3H, m), 6.17-6.24 (1H, m), 4.58-4.68 (2H, d), 4.30-4.40 (4H, m), 3.46 (2H, s), 1.32-1.40 (3H, t) ppm.

LRMS (electrospray): m/z [M+H]⁺ 540, [M+Na]⁺ 562, [M−H]⁺ 538.

The following Preparations describe the preparation of certain intermediates used in the preceding Examples.

Preparation 1: (4-Aminomethyl-benzyl)-carbamic acid tert-butyl ester

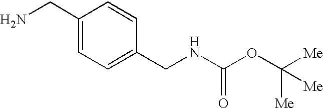

A solution of di-tert-butyl-dicarboxylate (4.62 g, 21.2 mmol) dissolved in dichloromethane (50 ml) was added to a solution of 4-aminomethyl-benzylamine (14.4 g, 106 mmol) in dichloromethane (50 ml) at 0° C. under an atmosphere of nitrogen. The reaction mixture was allowed to warm to room temperature and stirred for 18 hours.

The reaction mixture was then washed sequentially with water (100 ml) and a 10% aqueous solution of citric acid (200 ml) and the organic phase disgarded. The pH of the aqueous phase was then adjusted to pH higher than 8 by addition of 0.88 ammonia and extracted with dichloromethane (3-fold 200 ml). The combined organic extracts were then dried over anhydrous magnesium sulphate and the solvent removed in vacuo giving (4-aminomethyl-benzyl)-carbamic acid tert-butyl ester (4.29 g) as a white solid.

¹H NMR (400 MHz, CDCl₃): δ=7.22-7.26 (4H, d), 4.80-4.90 (1H, brs), 4.23-4.30 (2H, m), 3.82 (2H, s), 1.43 (2H, s), 1.38 (2H, s) ppm.

LRMS (electrospray): m/z [M−H]⁺ 237.

Preparation 2: [4-({[2-(4-Fluoro-phenoxy)-pyridine-3-carbonyl]-amino}-methyl)-benzyl]-carbamic acid tert-butyl ester

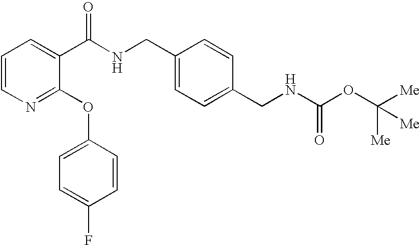

2-(4-Fluoro-phenoxy)-nicotinic acid (see reference Patent application WO 98/45268) (6.20 g, 26 mmol), 1-hydroxybenzotriazole (5.39 g, 40 mmol) and 1-(3-dimethylaminopropyl)-3-ethylcarbodiimide hydrochloride (6.62 g, 34 mmol) were disolved in N,N-dimethylformamide (50 ml) at room temperature and (4-aminomethyl-benzyl)-carbamic acid tert-butyl ester (6.28 g, 26 mmol) (see Preparation 1) added followed by addition of N-methyl morpholine (4.4 ml, 40 mmol). The reaction mixture was stirred under an atmosphere of nitrogen at room temperature for 18 hours, and then partitioned between ethyl acetate (100 ml) and water (100 ml) and the organic layer separated. The organic phase was then washed with a saturated aqueous solution of sodium chloride (100 ml), dried over anhydrous magnesium sulphate and the solvent removed in vacuo. The residue was triturated with diethylether (15 ml) giving [4-({[2-(4-fluoro-phenoxy)-pyridine-3-carbonyl]-amino}-methyl)-benzyl]-carbamic acid tert-butyl ester (9.52 g) as an off-white solid.

¹H NMR (300 MHz, CDCl₃): δ=8.56-8.76 (1H, m), 8.06-8.14 (2H, m), 6.96-7.40 (9H, m, partialy masked by solvent), 4.58-4.95 (3H, m), 4.20-4.40 (2H, brs), 1.56 (9H, s) ppm.

LRMS (thermospray): m/z [M+NH₄]⁺ 469.

Preparation 3: N-(4-Aminomethyl-benzyl)-2-(4-fluoro-phenoxy)-nicotinamide hydrochloride

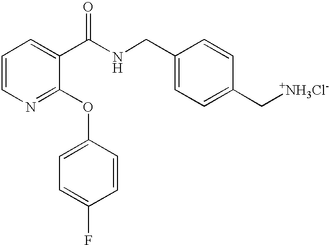

[4-({[2-(4-Fluoro-phenoxy)-pyridine-3-carbonyl]-amino}-methyl)-benzyl]-carbamic acid tert-butyl ester (9.51 g, 21 mmol) (see Preparation 2) was dissolved in dichloromethane (60 ml) and hydrogen chloride gas bubbled into the solution at 0° C. until the solution became saturated (30 minutes). The reaction mixture was then stirred under an atmosphere of nitrogen at room temperature for a further 1 hour before removal of the solvent in vacuo. The resultant white precipitate was triturated with diethylether (3-fold 10 ml) giving N-(4-aminomethyl-benzyl)-5-fluoro-2-(4-fluoro-phenoxy)-nicotinamide hydrochloride (7.92 g) as a white solid.

¹H NMR (300 MHz, DMSO-d⁶): δ=8.96-9.07 (1H, m), 8.40-8.60 (2H, m), 8.17-8.22 (1H, d), 8.11-8.16 (1H, m), 7.36-7.44 (4H, m), 7.18-7.33 (5H, m), 4.43-4.58 (2H, m, partially masked by solvent), 3.86-3.99 (2H, m) ppm.

LRMS (thermospray): m/z [M+H]⁺ 352.

Preparation 4: (4-{[2-Chloro-5-fluoro-pyridine-3-carbonyl)-amino]-methyl)-benzyl)-carbamic acid tert-butyl ester

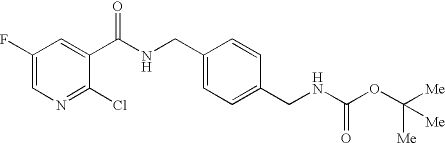

2-Chloro-5-fluoro-nicotinic acid (see Preparation 10) (2.0 g, 11.4 mmol), 1-hydroxybenzotriazole (1.85 g, 13.7 mmol) and 1-(3-dimethylaminopropyl)-3-ethylcarbodiimide hydrochloride (2.62 g, 13.7 mmol) were stirred in N,N-dimethylformamide (50 ml) at room temperature and (4-aminomethyl-benzyl)-carbamic acid tert-butyl ester (2.69 g, 11.4 mmol) (see Preparation 1) added followed by addition of N-methyl morpholine (2.5 ml, 22.8 mmol). The reaction mixture was then stirred under an atmosphere of nitrogen at room temperature for 18 hours, partitioned between dichloromethane (100 ml) and water (100 ml), and the organic layer separated. The organic layer was then washed with a saturated aqueous solution of sodium chloride (100 ml), dried over anhydrous magnesium sulphate and the solvent removed in vacuo to give (4-{[2-chloro-5-fluoro-pyridine-3-carbonyl)-amino]-methyl)-benzyl)-carbamic acid tert-butyl ester (4.08 g) as a white solid.

¹H NMR (400 MHz, DMSO-d⁶): δ=9.10-9.17 (1H, t), 8.52-8.54 (1H, d), 7.99-8.04 (1H, dd), 7.26-7.35 (3H, m), 7.18-7.22 (2H, d), 4.39-4.44 (2H, d), 4.06-4.11 (2H, d), 1.38 (9H, s) ppm.

LRMS (electrospray): m/z [M+Na]⁺ 416, [M−H]⁺ 392.

Preparation 5: [4-({[5-Fluoro-2-(4-fluoro-phenoxy)-pyridine-3-carbonyl]-amino}-methyl)-benzyl]-carbamic acid tert-butyl ester

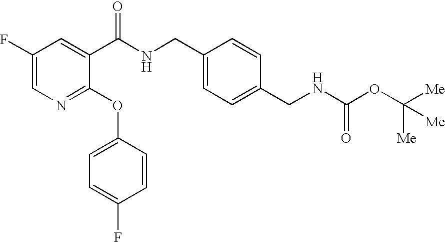

(4-{[2-Chloro-5-fluoro-pyridine-3-carbonyl)-amino]-methyl)-benzyl)-carbamic acid tert-butyl ester (100 mg, 0.29 mmol) (see Preparation 4), 4-fluorophenol (28 mg, 0.29 mmol) and caesium carbonate (800 mg, 2.5 mmol) were stirred in N,N-dimethylformamide (10 ml) at 60° C. under an atmosphere of nitrogen for 18 hours. The reaction mixture was then partitioned between ethyl acetate (20 ml) and water (20 ml), and the organic layer separated. The organic layer was then washed with a saturated aqueous solution of sodium chloride (3-fold 10 ml), the solvent removed in vacuo and the residue purified by flash column chromatography on silica gel eluting with a solvent gradient of 5:95 changing to 30:70, by volume, ethyl acetate : pentane to give [4-({[5-fluoro-2-(4-fluoro-phenoxy)-pyridine-3-carbonyl]-amino}-methyl)-benzyl]-carbamic acid tert-butyl ester (57 mg) as a white foam.

¹H NMR (400 MHz, DMSO-d⁶): δ=8.97-9.02 (1H, t), 8.19-8.21 (1H, d), 8.03-8.08 (1H, dd), 7.30-7.36 (1H, m), 7.19-7.30 (6H, m), 7.11-7.16 (2H, d), 4.44-4.50 (2H, d), 4.03-4.08 (2H, d), 1.36 (9H, s) ppm.

LRMS (electrospray): m/z [M+Na]⁺ 492, [M−H]⁺ 468.

Preparation 6: N-(4-Aminomethyl-benzyl)-5-fluoro-2-(4-fluoro-phenoxy)-nicotinamide hydrochloride

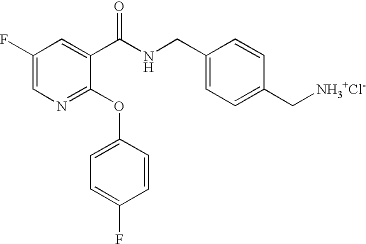

[4-({[5-Fluoro-2-(4-fluoro-phenoxy)-pyridine-3-carbonyl]-amino}-methyl)-benzyl]-carbamic acid tert-butyl ester (1.62 g, 3.44 mmol) (see Preparation 5) was dissolved in a 2.25 M solution of hydrochloric acid in methanol (100 ml) and the mixture stirred at room temperature under an atmosphere of nitrogen for 4 hours before removing the solvent in vacuo. The residue was dissolved in water (50 ml), the pH adjusted to pH higher than 8 by addition of sodium hydrogen carbonate and extracted with dichloromethane (3-fold 50 ml). The combined organic extracts were dried over anhydrous magnesium sulphate and concentrated in vacuo to give N-(4-aminomethyl-benzyl)-5-fluoro-2-(4-fluoro-phenoxy)-nicotinamide hydrochloride (1.25 mg) as a gum.

Preparation 7: (4-{[(2-Chloro-pyridine-3-carbonyl)-amino]-methyl}-benzyl)-carbamic acid tert-butyl ester

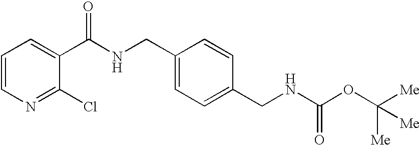

2-Chloro-nicotinic acid (2.86 g, 18.2 mmol), 1-hydroxybenzotriazole (3.0 g, 21.8 mmol) and 1-(3-dimethylaminopropyl)-3-ethylcarbodiimide hydrochloride (4.18 g, 21.8 mmol) were dissolved in N,N-dimethylformamide (50 ml) at room temperature and (4-aminomethyl-benzyl)-carbamic acid tert-butyl ester (4.29 g, 18.2 mmol) (see Preparation 1) added followed by addition of N-methyl morpholine (4 ml, 36.3 mmol). The reaction mixture was stirred under an atmosphere of nitrogen at room temperature for 18 hours, then partitioned between ethyl acetate (100 ml) and water (100 ml) and the organic layer separated. The organic layer was then washed with a saturated aqueous solution of sodium chloride (100 ml), dried over anhydrous magnesium sulphate and the solvent removed in vacuo. The residue was then triturated with diethylether (2-fold 10 ml) to give (4-{[(2-chloro-pyridine-3-carbonyl)-amino]-methyl}-benzyl)-carbamic acid tert-butyl ester (6.71 g) as a white solid.

¹H NMR (400 MHz, DMSO-d⁶): δ=9.01-9.08 (1H, t), 8.43-8.47 (1H, m), 7.89-7.93 (1H, d), 7.45-7.50 (1H, m), 7.30-7.37 (1H, m), 7.26-7.31 (2H, d), 7.17-7.21 (2H, d), 4.39-4.43 (2H, d), 4.03-4.10 (2H, d), 1.37 (9H, s) ppm.

LRMS (electrospray): m/z [M−H]⁺ 374.

Preparation 8: 3-{3-[4-tert-Butoxycarbonylamino-methyl)-benzylcarbamoyl]-pyridin-2-yloxy}-benzoic acid ethyl ester

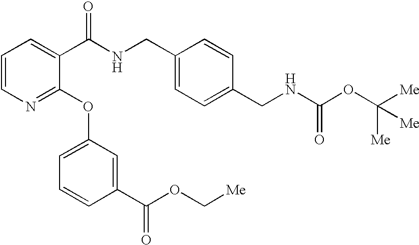

(4-{[(2-Chloro-pyridine-3-carbonyl)-amino]-methyl}-benzyl)-carbamic acid tert-butyl ester (12.0 g, 32.2 mmol) (see Preparation 7), 3-hydroxy-benzoic acid ethyl ester (6.42 g, 38.6 mmol) and caesium carbonate (15.7 g, 48.3 mmol) were stirred in dioxan (180 ml) at 70° C. under an atmosphere of nitrogen for 18 hours. Starting material remained, so a further aliquot of 3-hydroxy-benzoic acid ethyl ester (6.42 g, 38.6 mmol) and caesium carbonate (15.7 g, 48.3 mmol) were added along with dioxan (420 ml) and N,N-dimethylformamide (40 ml) and the reaction stirred at 70° C. for a further 22 hours. The solvent was then removed under reduced pressure, the residue partitioned between ethyl acetate (200 ml) and water (200 ml), and the organic layer separated. The organic layer was then washed with a saturated aqueous solution of sodium chloride (3-fold 100 ml), the solvent removed in vacuo and the residue purified by flash column chromatography on silica gel eluting with a solvent gradient of 0:100 changing to 50:50, by volume, ethyl acetate:hexane to give 3-{3-[4-tert-butoxycarbonylamino-methyl)-benzylcarbamoyl]-pyridin-2-yloxy}benzoic acid ethyl ester (7.42 mg) as an off-white foam.

¹H NMR (400 MHz, DMSO-d⁶): δ=8.92-8.98 (1H, t), 8.18-8.21 (1H, d), 8.14-8.18 (1H, d), 7.81-7.85 (1H, d), 7.77 (1H, s), 7.54-7.60 (1H, t), 7.46-7.50 (1H, m), 7.27-7.31 (2H, d), 7.22-7.26 (1H, m), 7.14-7.18 (3H, d), 4.47-4.51 (2H, d), 4.29-4.35 (2H, quart), 4.04-4.08 (2H, d), 1.37 (9H, s), 1.28-1.35 (3H, t) ppm.

LRMS (electrospray): m/z [M+Na]⁺ 528, [M−H]⁺ 504.

Preparation 9: [N-(4-Aminomethyl-benzyl)-2-(4-fluoro-phenoxy)]-nicotinamide hydrochloride

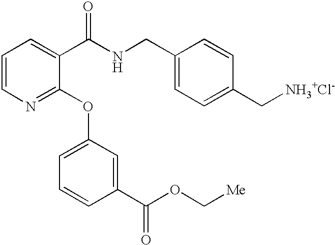

3-{3-[4-tert-Butoxycarbonylamino-methyl)-benzylcarbamoyl]-pyridin-2-yloxy}-benzoic acid ethyl ester (7.42 g, 14.7 mmol) (see Preparation 8) was dissolved in dichloromethane (100 ml) and hydrogen chloride gas bubbled through the solution at 0° C. until the solution became saturated (30 minutes). The solvent was removed in vacuo giving [N-(4-aminomethyl-benzyl)-2-(4-fluoro-phenoxy)]-nicotinamide hydrochloride (7.16 mg) as a white solid.

¹H NMR (400 MHz, DMSO-d⁶): δ=9.48-9.54 (1H, m), 8.83-9.03 (3H, brs), 8.62-8.66 (1H, m), 8.57-8.63 (1H, d), 8.35-8.42 (1H, d), 8.22 (1H, s), 8.01-8.08 (1H, t), 7.93-7.98 (1H, d), 7.81-7.91 (4H, m), 7.68-7.74 (1H, d), 4.94-5.01 (2H, d), 4.76-4.81 (2H, quart), 4.36-4.42 (2H, m), 1.75-1.80 (3H, t) ppm.

LRMS (electrospray): m/z [M+H]⁺ 406.

Preparation 10: 2-Chloro-5-fluoro nicotinic acid

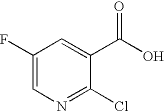

Ethyl-2-chloro-5-fluoro-nicotinoate (50.4 g, 0.247 mol) (see reference J. Med. Chem., 1993, 36(18), 2676-88) was dissolved in tetrahydrofuran (350 ml) and a 2 M aqueous solution of lithium hydroxide (247 ml, 0.495 mol) added. The reaction mixture was stirred at room temperature for 3 days. The pH of the solution was reduced to pH equal to 1 by addition of 6N hydrochloric acid and then extracted with dichloromethane. The combined extracts were dried over anhydrous magnesium sulphate and the solvent removed in vacuo to give a solid which was triturated with diethyl ether and then dried in vacuo to give 2-chloro-5-fluoro nicotinic acid (40.56 g) as a white solid.

¹H NMR (400 MHz, DMSO-d⁶): δ=8.20 (1H, s), 8.62 (1H, s) ppm.

LRMS (electrospray): m/z [M+H]⁺ 174.

In Vitro Activity of the Nicotinamide Derivatives

The PDE4 inhibitory activity of the nicotinamide derivatives of the formula (1) is determined by the ability of compounds to inhibit the hydrolysis of cAMP to AMP by PDE4 (see also reference 1). Tritium labelled cAMP is incubated with PDE4. Following incubation, the radiolabelled AMP produced is able to bind ytrium silicate SPA beads. These SPA beads subsequently produce light that can be quantified by scintillation counting. The addition of a PDE4 inhibitor prevents the formation of AMP from cAMP and counts are diminished. The IC₅₀ of a PDE4 inhibitor can be defined as the concentration of a compound that leads to a 50% reduction in counts compared to the PDE4 only (no inhibitor) control wells.

The anti-inflammatory properties of the nicotinamide derivatives of the formula (1) are demonstrated by their ability to inhibit TNFα release from human peripheral blood mononuclear cells (see also reference 2). Venous blood is collected from healthy volunteers and the mononuclear cells purified by centrifugation through Histopaque (Ficoll) cushions. TNFα production from these cells is stimulated by addition of lipopolysaccharide. After 18 hours incubation in the presence of LPS, the cell supernatant is removed and the concentration of TNFα in the supernatant determined by ELISA. Addition of PDE4 inhibitors reduces the amount of TNFα produced. An IC₅₀ is determined which is equal to the concentration of compound that gives 50% inhibition of TNFα production as compared to the LPS stimulated control wells.

All the examples were tested in the assay described above and found to have an IC₅₀ (TNFα screen) of less than 500 nM. And for most of the tested compounds, they were found to have an IC₅₀ (TNFα screen) of even less than 200 nM.

References:

1. Thompson J W, Teraski W L, Epstein P M, Strada S J., “Assay of nucleotidephosphodiesterase and resolution of multiple molecular forms of the isoenzyme”, Advances in cyclic nucleotides research, edited by Brooker G, Greengard P, Robinson G A. Raven Press, New York 1979, 10, p. 69-92.

2. Yoshimura T, Kurita C, Nagao T, Usami E, Nakao T, Watanabe S, Kobayashi J, Yamazaki F, Tanaka H, Nagai H., “Effects of cAMP-phosphodiesterase isozyme inhibitor on cytokine production by lipopolysaccharide-stimulated human peripheral blood mononuclear cells”, Gen. Pharmacol., 1997, 29(4), p. 63

1. A combination of a compound of the formula (1): 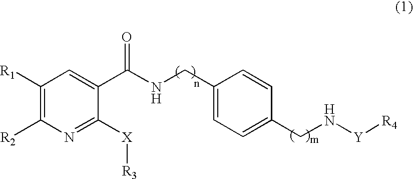
wherein:
m is 0, 1, 2 or 3;
n is 0, 1, 2 or 3;
R₁ and R₂ are each independently hydrogen, halo, cyano, (C₁-C₄)alkyl or (C₁-C₄)alkoxy;
X is —O—, —S— or —NH—;
R₃ is:
(a) phenyl, naphthyl, heteroaryl or (C₃-C₈)cycloalkyl, each optionally substituted independently with 1 to 3 halo, cyano, (C₁-C₄)alkyl, (C₁-C₄)alkoxy, (C₁-C₄)thioalkyl, —C(═O)NH₂, —C(═O)NH((C₁-C₄)alkyl), hydroxy, —O—C(═O)(C₁-C₄)alkyl, —C(═O)—O—(C₁-C₄)alkyl or hydroxy(C₁-C₄)alkyl; or
(b) a bicyclic group of the following formula: 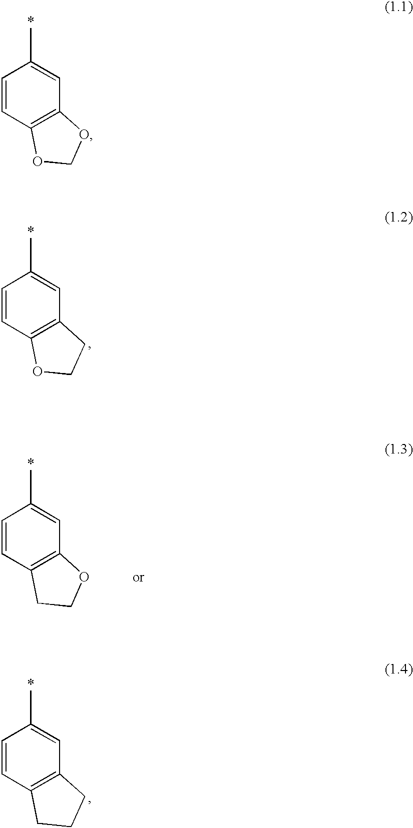
where the symbol “*” in the definition of R₃ indicates the point of attachment of each partial formula (1.1) through (1.4) to the remaining portion of formula (1);
Y is 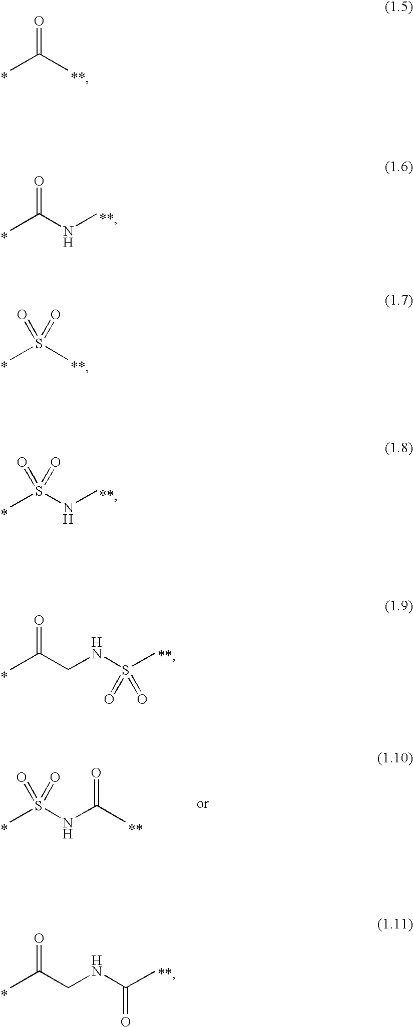
where the symbol “*” in the definition of Y indicates the point of attachment of each partial formula (1.5) through (1.11) to the remaining portions —NH— of formula (1) and “**” in the definition of Y indicates the point of attachment of each partial formula (1.5) through (1.11) to the remaining portions —R₄ of formula (1); and
R₄ is:
(a) phenyl, naphthyl or heteroaryl, each optionally substituted independently with 1 to 3 carboxy, —C(═O)—O—(C₁-C₄)alkyl, halo, cyano, —C(═O)NH₂, (C₁-C₄)alkyl, (C₁-C₄)alkoxy, (C₁-C₄)haloalkyl, hydroxy or hydroxy(C₁-C₄)alkyl; or
(b) (C₁-C₄)alkyl optionally substituted with hydroxy, carboxyl, C(═O)—O—(C₁-C₄)alkyl, phenyl, naphthyl or heteroaryl wherein said phenyl, naphthyl and heteroaryl are each optionally substituted independently with 1 to 3 carboxy, —C(═O)O(C₁-C₄)alkyl, halo, cyano, —C(═O)NH—₂, (C₁-C₄)alkyl, (C₁-C₄)alkoxy, (C₁-C₄)haloalkyl, hydroxy or hydroxy(C₁-C₄)alkyl; or a pharmaceutically acceptable salt, isomer, tautomer, solvate, polymorph, isotopic variation or metabolite thereof with tiotropium or a derivative thereof.

2. A combination according to claim 1 except the combinations with a compound of formula (1) for which:
1) m is different from 0 simultaneously with Y representing the partial formula (1.5) and R₄ representing a non-substituted (C₁-C₄)alkyl,
2) m is equal to 0 simultaneously with Y representing the partial formula (1.5) and R₄ representing a phenyl, a naphthyl or a heteroaryl each optionally substituted with 1 to 3 substituents independently selected from the group consisting of carboxylic acid, halo, cyano, (C₁-C₄)alkyl, (C₁-C₄)alkoxy, (C₁-C₄)haloalkyl, hydroxy and hydroxy(C₁-C₄)alkyl or R₄ representing a (C₁-C₄)alkyl optionally substituted with a hydroxy, carboxylic acid, or a heteroaryl, which is optionally substituted with 1 to 3 substituents independently selected from the group consisting of carboxylic acid, halo, cyano, (C₁-C₄)alkyl, (C₁-C₄)alkoxy, hydroxy and hydroxy(C₁-C₄)alkyl, and
3) m is equal to 0 simultaneously with Y representing the partial formula (1.6) and R₄ representing a phenyl or a naphtyl, each optionally substituted with 1 to 3 substituents independently selected from the group consisting of carboxylic acid, halo, cyano, (C₁-C₄)alkyl, (C₁-C₄)alkoxy, (C₁-C₄)haloalkyl, hydroxy and hydroxy(C₁-C₄)alkyl.

3. A combination according to claim 1 wherein for the compound of formula (1):
m and n are each 1;
R₁ and R₂ are each independently hydrogen, halo, cyano, (C₁-C₄)alkyl or (C₁-C₄)alkoxy;
X is —O—;
R₃ is:
(a) phenyl, naphthyl, heteroaryl or (C₃-C₈)cycloalkyl, each optionally substituted independently with 1 to 3 halo, cyano, (C₁-C₄)alkyl, (C₁-C₄)alkoxy, (C₁-C₄)thioalkyl, —C(═O)NH₂, —C(═O)NH((C₁-C₄)alkyl), hydroxy, —O—C(═O)(C₁-C₄)alkyl, —C(═O)—O—(C₁-C₄)alkyl or hydroxy(C₁-C₄)alkyl; or
(b) a bicyclic group of the formula: 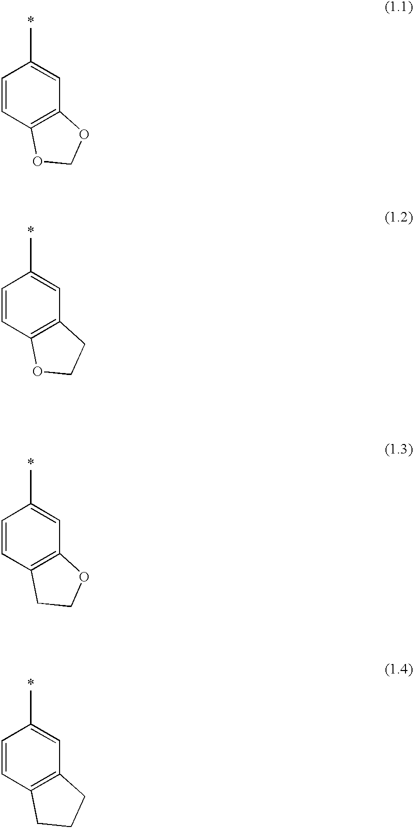
where the symbol “*” in the definition of R₃ indicates the point of attachment of each partial formula (1.1) through (1.4) to the remaining portion of formula (1);
Y is —C(═O)—; and
R₄ is:
(a) phenyl, naphthyl or heteroaryl, each optionally substituted independently with 1 to 3 carboxy, —C(═O)—O—(C₁-C₄)alkyl, halo, cyano, —C(═O)NH₂, (C₁-C₄)alkyl, (C₁-C₄)alkoxy, (C₁-C₄)haloalkyl, hydroxy and hydroxy(C₁-C₄)alkyl; or
(b) (C₁-C₄)alkyl substituted independently with a hydroxy, carboxylic acid, C(═O)—O—(C₁-C₄)alkyl, phenyl, naphthyl or heteroaryl group wherein said phenyl, naphthyl and heteroaryl are each optionally substituted with 1 to 3 substituents each selected from the group consisting of carboxylic acid, C(═O)O(C₁-C₄)alkyl, halo, cyano, —C(═O)NH₂, (C₁-C₄)alkyl, (C₁-C₄)alkoxy, (C₁-C₄)haloalkyl, hydroxy, and hydroxy(C₁-C₄)alkyl, or a pharmaceutically acceptable salt, isomer, tautomer, solvate, polymorph, isotopic variation or metabolite thereof.

4. A combination according to claim 1 wherein for the compound of formula (1):
m and n are each 1;
R₁ and R₂ are each independently hydrogen, halo and methyl;
X is —O—;
R₃ is a phenyl optionally substituted independently with 1 to 3 halo, cyano, (C₁-C₄)alkyl, (C₁-C₄)alkoxy, (C₁-C₄)thioalkyl, —C(═O)NH₂, —C(═O)NH((C₁-C₄)alkyl), hydroxy, —O—C(═O)(C₁-C₄)alkyl,
—C(═O)—O—(C₁-C₄)alkyl or hydroxy(C₁-C₄)alkyl;
Y is —C(═O)—; and
R₄ is:
(a) phenyl optionally substituted independently with 1 to 3 carboxy, —C(═O)—O—(C₁-C₄)alkyl, halo, cyano, —C(═O)NH₂, (C₁-C₄)alkyl, (C₁-C₄)alkoxy, (C₁-C₄)haloalkyl, hydroxy or hydroxy(C₁-C₄)alkyl; or
(b) (C₁-C₄)alkyl substituted with hydroxy or phenyl, wherein said phenyl is optionally substituted independently with 1 to 3 carboxy, —C(═O)O(C₁-C₄)alkyl, halo, cyano, —C(═O)NH₂, (C₁-C₄)alkyl or (C₁-C₄)alkoxy, (C₁-C₄)haloalkyl, hydroxy or hydroxy(C₁-C₄)alkyl;
or a pharmaceutically acceptable salt, isomer, tautomer, solvate, polymorph, isotopic variation or metabolite thereof.

5. A combination according to claim 1 wherein for the compound of formula (1):
m and n are each 1;
R₁ is hydrogen or fluoro and R₂ is hydrogen;
X is —O—;
R₃ is phenyl optionally substituted with halo or —C(═O)—O—(C₁-C₄)alkyl;
Y is —C(═O)—; and
R₄ is:
(a) phenyl optionally substituted independently with 1 to 3 halo, (C₁-C₄)alkyl or hydroxy; or
(b) (C₁-C₄)alkyl substituted with hydroxy or phenyl, wherein said phenyl is optionally substituted independently with 1 to 3 halo, (C₁-C₄)alkyl and hydroxy;
or a pharmaceutically acceptable salt, isomer, tautomer, solvate, polymorph, isotopic variation or metabolite thereof.

6. A combination according to claim 1 wherein the compound of formula (1) is selected from the group consisting of:
2-(4-fluoro-phenoxy)-N-{4-[(2-hydroxy-3-methyl-benzoyl amino)-methyl]-benzyl}-nicotinamide;
3-(3-{4-[(3-hydroxy-benzoylamino)-methyl]-benzyl carbamoyl}-pyridin-2-yloxy)-benzoic acid ethyl ester;
2-(4-fluoro-phenoxy)-N-{4-[(6-fluoro-2-hydroxy-benzoylamino)-methyl]-benzyl}-nicotinamide;
2-(4-fluoro-phenoxy)-N-{4-[(5-fluoro-2-hydroxy-benzoylamino)-methyl]-benzyl}-nicotinamide;
2-(4-fluoro-phenoxy)-N-{4-[(3-hydroxy-4-methyl-benzoylamino)-methyl]-benzyl}-nicotinamide;
2-(4-fluoro-phenoxy)-N-{4-[(3-hydroxy-benzoylamino)-methyl]-benzyl}-nicotinamide;
2-(4-fluoro-phenoxy)-N-{4-[(2-hydroxy-benzoylamino)-methyl]-benzyl}-nicotinamide;
2-(4-fluoro-phenoxy)-N-{4-[(4-hydroxy-benzoylamino)-methyl]-benzyl}-nicotinamide;
2-(4-fluoro-phenoxy)-N-{4-[(2-hydroxy-4-methyl-benzoylamino)-methyl]-benzyl}-nicotinamide;
2-(4-fluoro-phenoxy)-N-{4-[(3-hydroxy-2-methyl-benzoylamino)-methyl]-benzyl}-nicotinamide;
2-(4-fluoro-phenoxy)-N-{4-[(2-hydroxy-5-methyl-benzoylamino)-methyl]-benzyl}-nicotinamide;
5-fluoro-2-(4-fluoro-phenoxy)-N-{4-[(2-hydroxy-benzoylamino)-methyl]-benzyl}-nicotinamide;
5-fluoro-2-(4-fluoro-phenoxy)-N-{4-[(2-hydroxy-acetyl-amino)-methyl]-benzyl}-nicotinamide;
5-fluoro-2-(4-fluoro-phenoxy)-N-{4-[(4-hydroxy-benzoylamino)-methyl]-benzyl}-nicotinamide;
3-(3-{4-[(3-hydroxy-benzoylamino)-methyl]-benzylcarbamoyl}-pyridin-2-yloxy)-benzoic acid ethyl ester;
3-(3-{4-[(2-hydroxy-phenacetyl-amino)-methyl]-benzylcarbamoyl}-pyridin-2-yloxy)-benzoic acid ethyl ester;
3-(3-{4-[(3-hydroxy-phenacetyl-amino)-methyl]-benzylcarbamoyl}-pyridin-2-yloxy)-benzoic acid ethyl ester; or
3-(3-{4-[(4-hydroxy-phenacetyl-amino)-methyl]-benzylcarbamoyl}-pyridin-2-yloxy)-benzoic acid ethyl ester.

7. A pharmaceutical composition comprising a compound of formula (1) 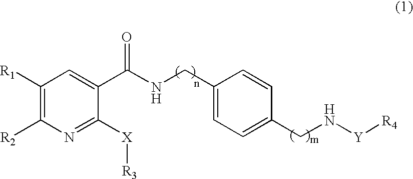
wherein:
m is 0, 1, 2 or 3;
n is 0, 1, 2 or 3;
R₁ and R₂ are each independently hydrogen, halo, cyano, (C₁-C₄)alkyl or (C₁-C₄)alkoxy;
X is —O—, —S— or —NH—;
R₃ is:
(a) phenyl, naphthyl, heteroaryl or (C₃-C₈)cycloalkyl, each optionally substituted independently with 1 to 3 halo, cyano, (C₁-C₄)alkyl, (C₁-C₄)alkoxy, (C₁-C₄)thioalkyl, —C(═O)N H₂, —C(═O)NH((C₁-C₄)alkyl), hydroxy, —O—C(═O)(C₁-C₄)alkyl, —C(═O)—O—(C₁-C₄)alkyl or hydroxy(C₁-C₄)alkyl; or
(b) a bicyclic group of the following formula: 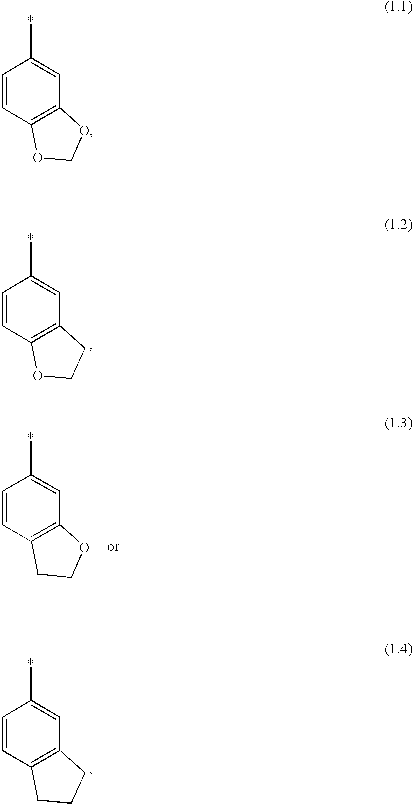
where the symbol “*” in the definition of R₃ indicates the point of attachment of each partial formula (1.1) through (1.4) to the remaining portion of formula (1);
Y is 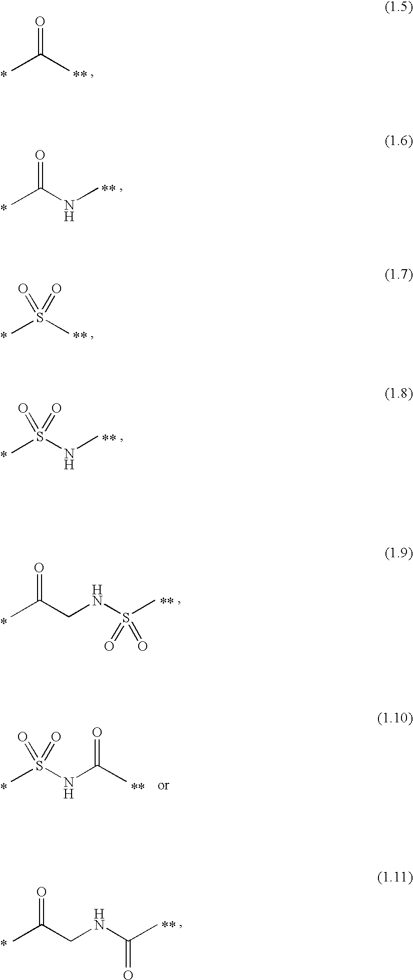
where the symbol “*” in the definition of Y indicates the point of attachment of each partial formula (1.5) through (1.11) to the remaining portions —NH— of formula (1) and “**” in the definition of Y indicates the point of attachment of each partial formula (1.5) through (1.11) to the remaining portions —R₄ of formula (1); and
R₄ is:
(a) phenyl, naphthyl or heteroaryl, each optionally substituted independently with 1 to 3 carboxy, —C(═O)—O—(C₁-C₄)alkyl, halo, cyano, —C(═O)NH₂, (C₁-C₄)alkyl, (C₁-C₄)alkoxy, (C₁-C₄)haloalkyl, hydroxy or hydroxy(C₁-C₄)alkyl; or
(b) (C₁-C₄)alkyl optionally substituted with hydroxy, carboxyl, C(═O)—O—(C₁-C₄)alkyl, phenyl, naphthyl or heteroaryl wherein said phenyl, naphthyl and heteroaryl are each optionally substituted independently with 1 to 3 carboxy, —C(═O)O(C₁-C₄)alkyl, halo, cyano, —C(═O)NH₂, (C₁-C₄)alkyl, (C₁-C₄)alkoxy, (C₁-C₄)haloalkyl, hydroxy or hydroxy(C₁-C₄)alkyl; or a pharmaceutically acceptable salt thereof, tiotropium or a derivative thereof and a pharmaceutically acceptable excipient and/or additive.

8. A pharmaceutical composition comprising a combination of claim 1.

9. A method of treating a disease, disorder or condition mediated by the PDE4 isozyme in a mammal, said method comprising administering to said mammal in need of such mediation, a therapeutically effective amount of a compound of formula (1) 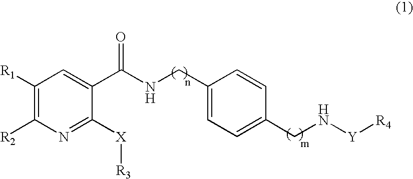
wherein:
m is 0, 1, 2 or 3;
n is 0, 1, 2 or 3;
R₁ and R₂ are each independently hydrogen, halo, cyano, (C₁-C₄)alkyl or (C₁-C₄)alkoxy;
X is —O—, —S— or —NH—;
R₃ is:
(a) phenyl, naphthyl, heteroaryl or (C₃-C₈)cycloalkyl, each optionally substituted independently with 1 to 3 halo, cyano, (C₁-C₄)alkyl, (C₁-C₄)alkoxy, (C₁-C₄)thioalkyl, —C(═O)N H₂, —C(═O)NH((C₁-C₄)alkyl), hydroxy, —O—C(═O)(C₁-C₄)alkyl, —C(═O)—O—(C₁-C₄)alkyl or hydroxy(C₁-C₄)alkyl; or
(b) a bicyclic group of the following formula: 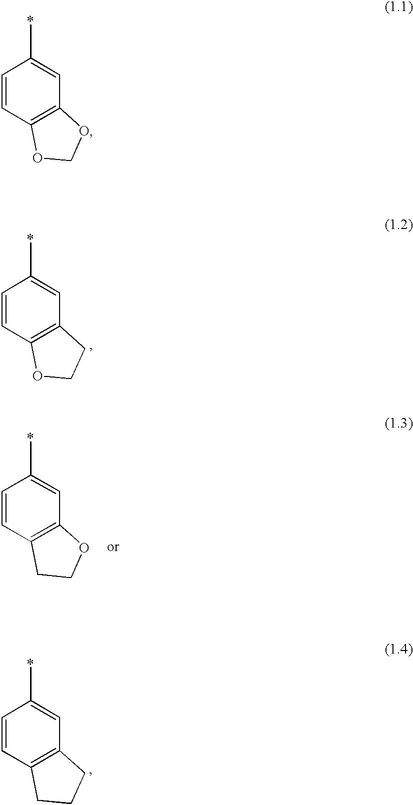
where the symbol “*” in the definition of R₃ indicates the point of attachment of each partial formula (1.1) through (1.4) to the remaining portion of formula (1);
Y is 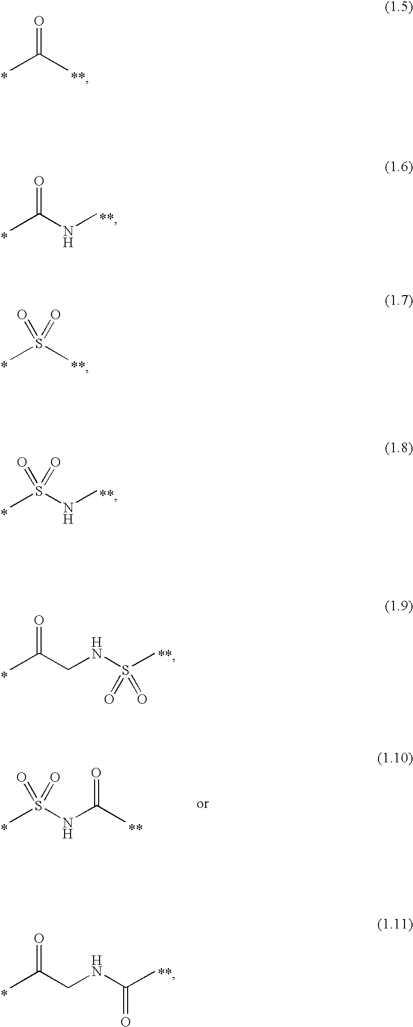
where the symbol “*” in the definition of Y indicates the point of attachment of each partial formula (1.5) through (1.11) to the remaining portions —NH— of formula (1) and “**” in the definition of Y indicates the point of attachment of each partial formula (1.5) through (1.11) to the remaining portions —R₄ of formula (1); and
R₄ is:
(a) phenyl, naphthyl or heteroaryl, each optionally substituted independently with 1 to 3 carboxy, —C(═O)—O—(C₁-C₄)alkyl, halo, cyano, —C(═O)NH₂, (C₁-C₄)alkyl, (C₁-C₄)alkoxy, (C₁-C₄)haloalkyl, hydroxy or hydroxy(C₁-C₄)alkyl; or
(b) (C₁-C₄)alkyl optionally substituted independently with hydroxy, carboxyl, C(═O)—O—(C₁-C₄)alkyl, phenyl, naphthyl or heteroaryl wherein said phenyl, naphthyl and heteroaryl are each optionally substituted independently with 1 to 3 carboxy, —C(═O)O(C₁-C₄)alkyl, halo, cyano, —C(═O)NH₂, (C₁-C₄)alkyl, (C₁-C₄)alkoxy, (C₁-C₄)haloalkyl, hydroxy or hydroxy(C₁-C₄)alkyl; or a pharmaceutically acceptable salt thereof and tiotropium or a derivative thereof.

10. A method of claim 9 wherein said disease, disorder or condition is asthma.

11. A method of claim 10 wherein said disease, disorder or condition is atopic asthma; non-atopic asthma; allergic asthma; bronchial asthma; essential asthma; true asthma; intrinsic asthma caused by pathophysiologic disturbances; extrinsic asthma caused by environmental factors; essential asthma of unknown or inapparent cause; bronchitic asthma; emphysematous asthma; exercise-induced asthma; occupational asthma; infective asthma caused by bacterial, fungal, protozoal or viral infection; non-allergic asthma; incipient asthma; or wheezy infant syndrome.

12. A method of claim 9 wherein said disease, disorder or condition is chronic or acute bronchoconstriction; chronic bronchitis; small airways obstruction; emphysema; pneumoconiosis; chronic eosinophilic pneumonia; chronic obstructive pulmonary disease; adult respiratory distress syndrome; or exacerbation of airways hyper-reactivity consequent to other drug therapy.

13. A method of claim 12 wherein said chronic obstructive pulmonary disease is characterized by irreversible, progressive airways obstruction.

14. A method of claim 12 wherein said pneumonconiosis is aluminosis; bauxite workers' disease; anthracosis; miners' disease; asbestosis; steam-fitters' asthma; chalicosis; flint disease; ptilosis caused by inhaling the dust from ostrich feathers; siderosis caused by the inhalation of iron particles; silicosis; grinders' disease; byssinosis; cotton-dust asthma; or talc pneumoconiosis.

15. A method of claim 9 wherein said disease, disorder or condition is bronchitis; acute bronchitis; chronic bronchitis; acute laryngotracheal bronchitis; arachidic bronchitis; catarrhal bronchitis; croupus bronchitis; dry bronchitis; infectious asthmatic bronchitis; productive bronchitis; staphylococcus bronchitis; streptococcal bronchitis; or vesicular bronchitis.

16. A method of claim 9 wherein said disease, disorder or condition is bronchiectasis; cylindric bronchiectasis; sacculated bronchiectasis; fusiform brochiectasis; capillary bronchiectasis; cystic bronchiectasis; dry bronchiectasis or follicular bronchiectasis.

17. A method of claim 9 wherein said disease, disorder or condition is seasonal allergic rhinitis; perennial allergic rhinitis; sinusitis; purulent sinusitis; nonpurulent sinusitis; acute sinusitis; chronic sinusitis; ethmoid sinusitis; frontal sinusitis; or sphenoid sinusitis.

18. A method of claim 9 wherein said disease, disorder or condition is regulated by the activation and degranulation of eosinophils.

19. A method of any one of claims 9-18 wherein said compound of claim 1 or pharmaceutically acceptable salt thereof and tiotropium or derivative thereof is administered together with a pharmaceutically acceptable excipient and/or additive.
